(19) United States
(12) Patent Application Publication
(10) Pub. No.: US 2023/0293049 A1
(43) Pub. Date: Sep. 21, 2023

(54) SYSTEMS AND METHODS FOR MONITORING RESPIRATION OF AN INDIVIDUAL

(71) Applicant: Cornell University, Ithaca, NY (US)

(72) Inventors: Julianne Imperato-McGinley, New York, NY (US); Ana C. Krieger, Rye, NY (US); Edwin C. Kan, Ithaca, NY (US)

(21) Appl. No.: 18/017,344

(22) PCT Filed: Jul. 28, 2021
(86) PCT No.: PCT/US2021/043570

§ 371 Date: Jan. 20, 2023

Related U.S. Application Data
(60) Provisional application No. 63/058,436, filed on Jul. 29, 2020.

(51) Int. Cl. A61B 5/113; A61B 5/05; A61B 5/11; A61B 5/00

(52) U.S. Cl. CPC A61B 5/113; A61B 5/05; A61B 5/1126; A61B 5/7267; A61B 5/7275; A61B 5/4818; A61B 5/7282; A61B 5/746; A61B 5/14542

(57) ABSTRACT

Methods and systems are provided for monitoring respiration of an individual. A first radiofrequency (“RF”) sensing signal is provided within a near-field coupling range of a respiratory motion to be measured to generate a respiratory measurement signal as the first RF sensing signal modulated by the respiratory motion. A respiratory measurement signal is detected. The respiratory motion is measured based on the respiratory measurement signal. A respiratory event is detected using the measured respiratory motion. In some embodiments, the method further includes predicting a respiratory event of the individual using a machine learning classifier. The machine learning classifier may be trained using one or more respiratory features and/or one or more blood oxygen features.

### CROSS-REFERENCE TO RELATED APPLICATIONS

This application claims priority to U.S. Provisional Application No. 63/058,436, filed on Jul. 29, 2020, now pending, the disclosure of which is incorporated herein by reference.

### FIELD OF THE DISCLOSURE

The present disclosure relates to sensors for monitoring respiration, and more particularly, for detecting and/or predicting respiratory events of an individual.

### BACKGROUND OF THE DISCLOSURE

Drug overdose deaths are the number one cause of deaths for Americans under fifty years old. The number of deaths in the opioid crisis exceeds the deaths resulting from the Vietnam War and the AIDS epidemic respectively. Opioid-related deaths even surpass current U.S. motor vehicle accidents. The United States faces an economic burden of $78.5 billion a year from prescription opioid misuse alone—a figure that encompasses healthcare costs, productivity losses, and the cost of addiction treatment and criminal justice involvement. This epidemic has cost the U.S. more than a trillion dollars since 2001 and could exceed $500 billion in the next three years alone. The White House declared the opioid epidemic to be a public health emergency in late October 2017.

Opioid overdose deaths increased in all 50 states from 1999 to 2016. In 2017, drug overdose killed more than 70,000 Americans—of which more than 42,000 died specifically of an opioid overdose. These deaths include opioid-dependent newborns and infants following accidental drug consumption as 70% of opioids are stored within the reach of children.

Specifically in New York City (NYC), in 2016, a drug overdose occurred every seven hours. With 1,374 deaths confirmed that year, it represented a 46% increase from 2015. Fentanyl, an opioid with 50 to 100 times more painkilling potency than morphine, continues to be added to street drugs (including heroin and cocaine) and is driving the significant increase in the overdose rate. This rate increased across all demographic groups in nearly every borough of NYC. The South Bronx has the highest rate of drug overdose deaths with 37.1 deaths per 100,000 New Yorkers—a figure higher than the 2015 rate from West Virginia, the state with the highest age-adjusted opioid overdose death rate in the nation (36 deaths per 100,000 West Virginians). In 2017, New York State had the third highest raw number of drug overdose deaths of any state in the nation. For Hispanic and black persons, New York State ranked first and third, respectively, in terms of opioid overdose deaths by race/ethnicity in 2016. Men in NYC were nearly four times more likely to die of a drug overdose than women based on 2017 data.

Despite all local, city, state, and federal nationwide efforts, the opioid crisis in the United States marches on, and there remains a long-felt need for interventions to prevent opioid fatalities.

### BRIEF SUMMARY OF THE DISCLOSURE

In order to effectively deal with the opioid crisis in the United States, there is a strong need for monitoring and alerting systems to allow for timely intervention aimed at the prevention of overdose-related deaths. In the majority of cases, opioid-related deaths occur due to respiratory depression that progresses to respiratory arrest and asystole. In an aspect, the present disclosure provides an innovative and effective wearable device to detect respiratory events such as, for example, the respiratory depression that precedes respiratory arrest, and triggering an alert mechanism to designated parties (e.g., family, friends, and/or the Police Emergency Unit) allowing for rapid emergency response. Rapid response is a critical step in helping to reduce the number of opioid-related deaths. The current statistics reveal that close to 80% of opioid-related deaths occur in residential settings in which family, friends, or loved ones are unaware of the fatal overdose, and unable to intervene in a timely manner. In its 2018 report on the opioid crisis, the U.S. Office of the Surgeon General highlights the need to “ . . . implement health information technologies to promote efficiency, actionable information, and high-quality care.”

In some embodiments of the present disclosure, systems and methods are provided to predict the occurrence of a respiratory event.

The present disclosure may be embodied as a method for monitoring respiration of an individual. A first radiofrequency (“RF”) sensing signal is provided within a near-field coupling range of a respiratory motion to be measured to generate a respiratory measurement signal as the first RF sensing signal modulated by the respiratory motion. The method includes detecting the respiratory measurement signal. The respiratory motion is measured over a measurement period based on the respiratory measurement signal. A respiratory event is detected using the measured respiratory motion.

In some embodiments, the method further includes predicting a respiratory event of the individual using a machine learning classifier, wherein the respiratory event is a central apnea, an obstructive apnea, a mixed apnea, a hypopnea, or respiratory effort related arousal (RERA). The classifier may be trained using one or more of the following parameters determined from the measured respiratory motion: mean breathing rate, breathing rate standard deviation, breathing rate coefficient of variation (COV), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, power ratio, breathing cycle number (total number of detected breathing cycles in the specific epoch), and time duration that no peak is detected.

In some embodiments, the method further includes providing a second RF sensing signal within a near-field coupling range of a second motion to be measured to generate a second measurement signal. A second measurement signal is detected. The second motion is measured based on the second measurement signal. In an example, the respiratory motion may be a thoracic motion and the second motion may be an abdominal motion. The classifier may be further trained using a phase difference between the thoracic motion and the abdominal motion.

In some embodiments of the method, a blood oxygen level of the individual is measured over the measurement period. The classifier may be trained using one or more of mean blood oxygen level, standard deviation of the blood oxygen level, percentage of time blood oxygen level is greater than a threshold value, and mean skewness of the breathing rate.

In some embodiments, an alert signal is provided if a respiratory event is detected. In some embodiments, an alert signal is provided if a respiratory event is predicted. In some embodiments, an alert signal is provided if a respiratory event characteristic of respiratory failure is detected.

In some embodiments of the present disclosure, systems and methods are provided to characterize the sleep of an individual.

The present disclosure may be embodied as a system for monitoring respiration of an individual. The system includes a first signal source for generating a first RF sensing signal. A first antenna is in electrical communication with the first signal source. The first antenna is configured to be disposed within a near-field coupling range of a respiratory motion to be measured to generate a respiratory measurement signal as the first RF sensing signal modulated by the respiratory motion. The system includes a first receiver a first receiver for detecting the respiratory measurement signal. A processor is configured to detect a respiratory event using the respiratory measurement signal. The processor is configured to provide an alert signal if a respiratory event is detected. In some embodiments, the processor is configured to provide an alert signal if a respiratory event characteristic of respiratory failure is detected.

In some embodiments, the processor further includes a machine learning classifier configured to predict a respiratory event of the individual, wherein the respiratory event is a central apnea, an obstructive apnea, a mixed apnea, a hypopnea, or respiratory effort related arousal (RERA). For example, the may be trained using one or more of the following parameters determined from the measured respiratory motion: mean breathing rate, breathing rate standard deviation, breathing rate coefficient of variation (COV), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, power ratio, breathing cycle number (total number of detected breathing cycles in the specific epoch), and time duration that no peak is detected. The processor is further configured to provide an alert signal if a respiratory event is predicted.

In some embodiments, the system further includes a second signal source for generating a second RF sensing signal. A second antenna may be in electrical communication with the second signal source. The second antenna is configured to be disposed within a near-field coupling range of a second motion to be measured to generate a second measurement signal as the second sensing signal modulated by the second motion. The system may include a second receiver for detecting the second measurement signal. The processor may be further configured to detect the respiratory event using the second measurement signal.

The respiratory motion is a thoracic motion, the second motion is an abdominal motion, and the classifier is further trained using a phase difference between the thoracic motion and the abdominal motion.

In some embodiments, the system includes a blood oxygen sensor in communication with the processor. The classifier may be is trained using one or more of: mean blood oxygen level, standard deviation of the blood oxygen level, percentage of time blood oxygen level is greater than a threshold value, and mean skewness of the breathing rate.

### DESCRIPTION OF THE DRAWINGS

For a fuller understanding of the nature and objects of the disclosure, reference should be made to the following detailed description taken in conjunction with the accompanying drawings.

FIG. 1 is a diagram of a system according to an embodiment of the present disclosure.

FIGS. 2A-2C show NCS signals. FIG. 2A depicts the signal model of a near-field coherent sensing (NCS) multiplexing antenna on the human chest. The direct emission (blue arrow), the near-field coupling (purple) and the backscattering (red) will change the antenna characteristics that contain heart and lung motion information. FIG. 2B shows a CST Microwave Studio simulation of the chest antenna where the far-field radiation contains the breath and heartbeat waveforms that can be separated by the quadrature scheme without resorting to spectral filtering. FIG. 2C shows a CST simulation of the near-field coupling to the torso.

FIGS. 3A-3B show embodiments of NCS system components. FIG. 4A depicts an embodiment of a semi-active NCS system, wherein in some environments (e.g., outdoors), the tag operates in the active mode and is powered up by its on-board battery. The vital signs are digitally recorded and broadcast by standard protocols such as Wi-Fi with the tag ID. FIG. 3B depicts the semi-active NCS system of FIG. 3A in a passive mode (e.g., for indoor use) where a ceiling-mounted reader can wirelessly power up the sensing tag and retrieve the vital signs. This can also be useful where a battery of the tag is not adequately charged.

FIGS. 4A-4B show exemplary data from PSG and NCS devices. FIG. 4A shows sample raw data and respiratory signal collected using polysomnography (PSG). FIG. 4B shows breath characteristics of sample obstructive apnea (top) and central apnea (bottom) in PSG. FIG. 4C shows a preliminary comparison for sample extracted breath rates and respiratory volumes using PSG and NCS.

FIG. 5. Vital-sign comparison between NCS with a single chest tag and Hexoskin (Hx) with respiratory inductive plethysmography (RIP) thorax and abdomen belts in various autonomous breathing exercises: (top) NCS waveforms after demodulation; (middle) extracted respiratory volumes; (bottom) extracted breath rates.

FIGS. 6A-6C show the NCS raw waveforms, respiratory volume, breath rate, and heart rate vital sign waveforms for breathing simulating central apnea (FIG. 6A), simulating Cheyne-Stokes (FIG. 6B), and simulated depressed breathing (FIG. 6C).

FIG. 7 is a chart of a method according to an embodiment of the present disclosure.

FIG. 8 depicts normalized, filtered thorax and abdomen respiration waveforms during an isovolumetric maneuver by a participant.

FIGS. 9A-9D show example epochs labeled as “normal” (FIG. 9A), “hypopnea” (FIG. 9B), “Obstructive Sleep Apnea” (FIG. 9C), and “Central Sleep Apnea” (FIG. 9D).

FIG. 10 is a histogram showing the number of unique epochs for each patient.

FIGS. 11A-11B show the cross validation results using NCS data (FIG. 11A) and PSG data (FIG. 11B), for three classes.

FIGS. 11A-11B show the results of a sleep disorder classifier using NCS data (FIG. 11A) and PSG data (FIG. 11B), for seven classes.

FIGS. 12A-12B show the results of a sleep disorder classifier using NCS data (FIG. 12A) and PSG data (FIG. 12B).

FIGS. 13A-13B are graphs showing accuracy using a Leave One Group Out validation scheme (leaving one patient's data set as a test set and all other patients' data as a training set) for NCS data (FIG. 13A) and PSG data (FIG. 13B). Label criterion were: 1. no apnea and RERA classified as No Apnea; 2. snore classified as Snore; and 3. OSA, CSA, mixed apnea, hypopnea classified as Apnea.

FIGS. 14A-14B show the results of a disorder prediction classifier using NCS data (FIG. 14A) and PSG data (FIG. 14B), including SpO₂ features.

FIGS. 15A-15B show the results of another disorder prediction classifier using NCS data (FIG. 15A) and PSG data (FIG. 15B), excluding SpO₂ features.

FIGS. 16A-16B show the results of another disorder prediction classifier using NCS data (FIG. 16A) and PSG data (FIG. 16B), for three classes.

FIGS. 17A-17B show the results of another disorder prediction classifier using NCS data (FIG. 17A) and PSG data (FIG. 17B), for two classes.

FIG. 18 is a sleep characterization method according to another embodiment of the present disclosure.

FIGS. 19A-19B show example epochs labeled as “Sleep Stage N1” (FIG. 19A) and “Sleep Stage Wake” (FIG. 19B).

FIGS. 20A-20B show the results of a sleep stage classifier using NCS data (FIG. 20A) and PSG data (FIG. 20B), for four classes.

FIGS. 21A-21B show the results of another sleep stage classifier using NCS data (FIG. 21A) and PSG data (FIG. 21B), for three classes.

### DETAILED DESCRIPTION OF THE DISCLOSURE

Embodiments of the presently-disclosed NCS techniques are based on the principle of coupling vital signs to an RF antenna. A tiny movement inside the body such as a wrist pulse can be clearly and unambiguously retrieved from the antenna characteristics with established multiplexing. Preliminary testing on NCS shows the following useful characteristics for respiratory monitoring (such as detecting respiratory failure or respiratory arrest, predicting respiratory events, etc.):
No immediate skin contact or tension belts: Unlike an ECG, the NCS tag can be placed on top of, beneath, and/or within clothing in the form of, for example, a necklace, button, and bra ornament without the necessity of contact with the skin. Unlike RIP, no uncomfortable tension belt is required around the body. A wireless tag of the present device can be conveniently worn without operator help.
Least user motion constraint: An NCS tag in an active mode (e.g., self-powered) can retrieve vital signs reliably when the user moves freely, and the data transmission to external units is performed by standard digital protocols such as Wi-Fi, Bluetooth, etc. The NCS tag in the passive mode can tolerate most user motion with a ceiling-mounted or other nearby reader.
Multiplexing for multiple users: A reader in a room can read tens of passive tags simultaneously by, for example, code division multiple access (CDMA) with acceptable inter-tag interference. Multiple active tags and multiple readers can be added by existing wireless protocols or collaborative CDMA.
Cost-effective implementation: Embodiments of tags (e.g., semi-active tags) under mass production can cost less than $5, and can provide uninterrupted long-term operation for multiple free-motion users with literally no need of human operators. An indoor reader can cost less than $30 in view of today's RFID systems.
Equivalent accuracy: In initial testing on a number of subjects, the NCS accuracy with cardiopulmonary functions was comparable to RIP, ECG, and phonocardiogram (PCG).
Detailed waveform features: Due to the much lower noise floor, the NCS waveform features can be extracted for detailed pulmonary functions, heart rate variability, membrane dynamics, and airflow velocity.
Minimal RF radiation for user safety: In both passive and active modes, the RF energy coupled into the user body can be controlled to be much smaller than current standards, such as, for example, the current cellular phone safety standards, by using signal strength adaptation, duty-cycle control, etc.
Minimal interference to other wireless modules: The NCS operation has not only been conformed to present FCC wireless regulation, but also the required data rate is so low that long CDMA codes can be integrated into the output to minimize interference with other devices sharing the same wireless band.

In an aspect, the present disclosure may be embodied as a method for monitoring the respiration of an individual. For example, in some embodiments, the method may be used to detect a respiratory event, such as, for example, central apnea, an obstructive apnea, a mixed apnea, a hypopnea, or respiratory effort related arousal (RERA), respiratory depression, hypoventilation, or hypoxemia. A near-field coherent sensing (NCS) system is used to generate a respiratory measurement signal. Such an NCS system may couple a motion of interest to a radiofrequency sensing signal in the near field range. The “near field” is a region where induction characteristics dominate over radiation characteristics and the relationship between the electric field (E field) and the magnetic field (H field) is not well defined. In embodiments of the present disclosure, “near-field” may refer to the close-in region of an antenna where angular field distribution is dependent upon the distance from the antenna. In embodiments, the near-field extends to the region within one wavelength (λ) of the antenna. In other embodiments, the near-field extends to the region within λ/2, λ/3, λ/4, or λ/2π of the antenna, where λ is the operating wavelength of the antenna.

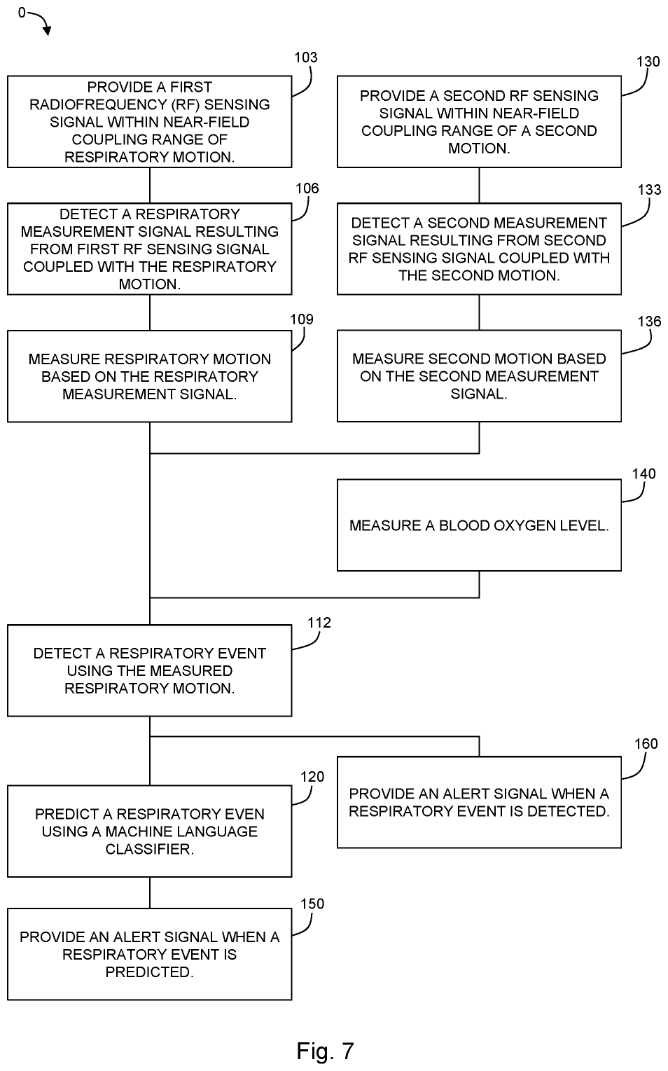

With reference to FIG. 7, the present disclosure may be embodied as a method 100 for monitoring respiration of an individual. The method 100 includes providing 103 a first radiofrequency (“RF”) sensing signal within a near-field coupling range of a respiratory motion to be measured to generate a respiratory measurement signal as the first RF sensing signal modulated by the respiratory motion. For example, a first antenna may be used to transmit a first RF sensing signal. A respiratory measurement signal is detected 106, where the respiratory measurement signal comprises the first RF sensing signal coupled with the respiratory motion of the individual. Continuing the previous example, a receiver may use the first antenna to receive the respiratory measurement signal as a reflected signal. In another example, a receiver may use an antenna located in the far field to detect the respiratory measurement signal as the coupled first RF sensing signal transmitted through the respiratory motion.

The provided first RF sensing signal may be an ID-modulated signal. In some embodiments, the first RF sensing signal is an active radio link. In some embodiments, the first RF sensing signal is a backscattered RFID link. For example, an antenna may emit a beacon or ID-modulated sensing signal in either an active radio link or a backscattering RFID (radio identification) link. The first RF sensing signal and the respiratory measurement signal may be matched using a cancellation network. In this way, any first RF sensing signal (i.e., non-modulated first RF sensing signal) detected with the first measurement signal may be reduced.

The respiratory motion is measured 109 based on the respiratory measurement signal. One or more parameters may be measured 109. For example, one or more of a breathing rate of the individual a peak-to-peak amplitude of the respiratory motion, an inhalation time, an exhalation time, a time duration during which no peak is detected (void time), a Power Ratio (further defined below), respiratory volume, and airflow (e.g., velocity). Such measurements may each include one or more of a mean, standard deviation, coefficient of variation (COV), skewness, kurtosis, and/or entropy of the measurement (e.g., the measurement over a period of time—the “measurement period”). A “Power Ratio” may be determined as a power spectral density of the respiratory measurement signal over a relevant bandwidth, divided by the power spectral density of the respiratory measurement signal over the entire measured bandwidth. An exemplary relevant bandwidth may be an expected range for respiration—for example, 0.05 Hz to 0.5 Hz.

The method 100 includes detecting 112 a respiratory event using the measured respiratory motion. For example, the method may include detecting respiratory depression (e.g., respiratory depression which may lead to respiratory failure (arrest)). The method may further include providing 160 an alert signal if a respiratory event characteristic of a respiratory failure of an opioid user is detected. For example, the alert signal may be one or more of an audible signal, a visual signal, an RF signal, an ultrasound signal, an infrared signal, a haptic signal, and the like. In some embodiments, a family member(s) or other caregivers may be notified through the use of an application, such as a smartphone app. Such family member(s) and/or caregivers may be provided with instructions on the use of the device, steps to be taken in case of respiratory depression, etc.

Some embodiments of the method 100 further include predicting 120 a respiratory event of the individual using a machine learning classifier. For example, a respiratory event such as a central apnea, an obstructive apnea, a mixed apnea, a hypopnea, or respiratory effort related arousal (RERA) before the occurrence of such an event. The machine learning classifier may be trained to detect the respiratory event or events of interest. The classifier may be trained using, for example, one or more parameters measured from the respiratory measurement signal (for example, as described above). Such parameters may include mean breathing rate, breathing rate standard deviation, breathing rate coefficient of variation (COV), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, Power Ratio, breathing cycle number (total number of detected breathing cycles in the specific epoch), and time duration that no peak is detected.

In some embodiments, a second RF sensing signal is provided 130 within a near-field coupling range of a second motion to be measured. In this way, the second RF signal is coupled with the second motion to generate a second measurement signal. The second measurement signal is detected 133. The second motion is measured 136 based on the second measurement signal. For example, the respiratory motion may be a thoracic motion (e.g., the NCS sensor located above (superior to) the xiphoid process) and the second motion may be an abdominal motion (e.g., the NCS sensor located below (inferior to) the xiphoid process). The machine learning classifier may be further trained using one or more parameters of the thoracic/abdominal relationship—e.g., a phase difference between the thoracic motion and the abdominal motion.

In some embodiments, the method 100 includes measuring 140 a blood oxygen level of the individual over the measurement period. The machine learning classifier may be trained using one or more of: mean blood oxygen level, standard deviation of the blood oxygen level, percentage of time blood oxygen level is greater than a threshold value (e.g., a predetermined threshold value), and mean skewness of the breathing rate.

An alert signal may be provided 150 when a respiratory event is predicted. For example, the alert signal may be one or more of an audible signal, a visual signal, an RF signal, an ultrasound signal, an infrared signal, a haptic signal, and the like. Such a signal may urge the individual to alter their sleep (e.g., sleep position) so as to avoid the predicted event. For example, a haptic signal may be provided to prompt the individual to switch sleep positions to avoid an occurrence of an obstructive apnea. In some embodiments, a family member(s) or other caregivers may be notified through the use of an application, such as a smartphone app.

In some embodiments, an event index may be created. For example, each respiratory event detected may be stored by a processor (e.g., in memory, in a file, etc.), along with the time of the event as an absolute time or relative time (e.g., elapsed time the event occurred during a sleep study). The stored data may be used to create an index score such as, for example, an Apnea-Hypopnea Index (AHI) score and/or a Respiratory Disturbance Index (RDI) score. For example, less than 5 events per hour may indicate a “normal” AHI or RDI, 5 to 14 events may be scored as “mild,” 15-30 may be scored as “moderate,” and more than 30 events per hour may be scored as “severe.”

Embodiments of the present disclosure utilize a near-field coherent sensing (NCS) system, which may be embodied as, for example, a sensing tag (e.g., a chest tag), able to capture the early cardiopulmonary signature of an opioid overdose. The wireless wearable detection system in the form of a sensing tag can be worn as a necklace, chest button, or other forms, to capture the early cardiopulmonary manifestations of a fatality. The present systems and methods are sufficiently accurate and robust to provide adequate non-invasive monitoring, while also providing comfort, convenience, and cost-effectiveness to reduce the number of opioid overdose deaths. The system provides sufficient accuracy and specificity to send an early alert to designated parties using a wireless network (e.g., Wi-Fi, smart phone, etc.) The detection of impending respiratory arrest with the NCS device with an alert will reduce deaths from opioid overdose and aid in combating this crisis in the United States.

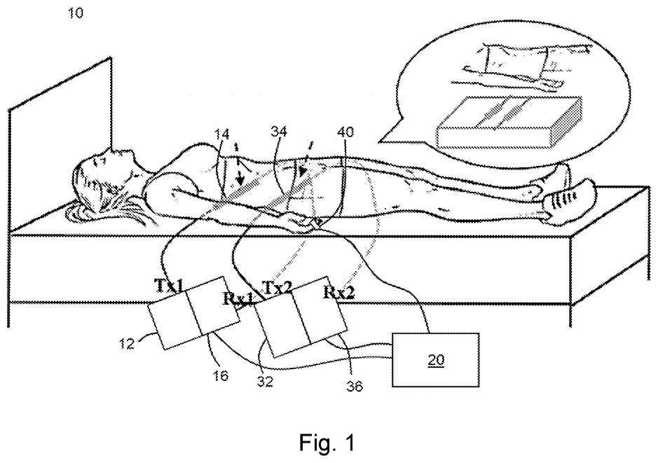

With reference to FIG. 1, in another aspect, the present disclosure may be embodied as a system 10 for monitoring respiration of an individual. The system 10 includes a first signal source 12 for generating a first RF sensing signal. A first antenna 14 is in electrical communication with the first signal source 12. The first antenna is configured to be disposed within a near-field coupling range of a respiratory motion to be measured. For example, the first antenna may be configured to be disposed within a coupling range of lung motion. In this way, a respiratory measurement signal is generated by the first RF sensing signal being modulated by the respiratory motion. The first RF sensing signal may be an ID-modulated wave. For example, the signal may be an active radio link or a backscattering RFID link.

The system 10 includes a first receiver 16 for detecting the respiratory measurement signal (the first RF sensing signal coupled with (i.e., modulated by) the respiratory motion) and a first Rx antenna in communication with the first receiver 16. The first receiver and/or the first Rx antenna may be configured to detect the respiratory measurement signal as a transmitted signal—i.e., far-field radiation. The first receiver and/or the first Rx antenna may be configured to detect the respiratory measurement signal as a reflected signal—i.e., antenna reflection. The system may include a filter in communication with the first receiver, wherein the filter is configured to demodulate and filter the respiratory measurement signal to obtain a motion signal. The filter may be, for example, a processor (such as a digital-signal processor (“DSP”)) programmed to sample, demodulate, and/or filter the respiratory measurement signal to derive the respiratory motion signal. The filter may be or may include a bandpass filter configured to filter the respiratory measurement signal using a first frequency range corresponding to the respiratory motion.

In some embodiments, the first RF sensing signal is a frequency-doubled downlink frequency. A Tx frequency doubler which doubles a downlink frequency before transmission via Tx antenna.

The system 10 further includes a processor 20 (e.g., a signal processing circuit, digital signal processor, microprocessor, discrete circuit, etc.) configured to use the measured respiratory motion to detect a respiratory event characteristic of respiratory failure (i.e., respiratory arrest) of an opioid user. In some embodiments, the processor may be configured to determine (i.e., calculate) a respiratory rate, determine a respiratory volume, and/or determine an airflow velocity. The processor may further be configured to provide an alert signal if a respiratory event characteristic of a respiratory failure of an opioid user is detected. For example, the alert signal may be one or more of an audible signal, a visual signal, an RF signal, an ultrasound signal, an infrared signal, a haptic signal, and the like.

The processor may include a machine learning classifier. The classifier may be configured to predict a respiratory event of the individual. For example, the respiratory event may be one or more of a central apnea, an obstructive apnea, a mixed apnea, a hypopnea, and/or respiratory effort related arousal (RERA). The classifier is trained using one or more of the following parameters determined from the measured respiratory motion: mean breathing rate, breathing rate standard deviation, breathing rate coefficient of variation (COV), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, power ratio, breathing cycle number (total number of detected breathing cycles in the specific epoch), and time duration that no peak is detected.

The system 10 may include a second signal source 32 for generating a second sensing signal. A second antenna 34 may be in electrical communication with the second signal source 32. The second antenna is configured to be disposed within a near-field coupling range of a second motion to be measured. For example, the second Tx antenna may be configured to be disposed within a coupling range of a heart motion, a pulse, an abdominal motion, a bowel motion, an eye motion, etc. In this way, a second measurement signal is generated by the second sensing signal being modulated by the second motion. The second sensing signal may be an ID-modulated wave. For example, the signal may be an active radio link or a backscattering RFID link. In some embodiments, the respiratory motion is a thoracic motion, the second motion is an abdominal motion, and the classifier is further trained using a phase difference between the thoracic motion and the abdominal motion.

The system 10 may further include a second receiver 36 for detecting the second measurement signal (the second sensing signal coupled with (i.e., modulated by) the second motion) and a second Rx antenna in communication with the second receiver. The second receiver and/or second first Rx antenna may be configured to detect the second measurement signal as a transmitted signal—i.e., far-field radiation. The second receiver and/or the second Rx antenna may be configured to detect the second measurement signal as a reflected signal—i.e., antenna reflection.

In some embodiments, the system 10 includes a blood oxygen sensor 40 in communication with the processor 20. The classifier may be trained using one or more of: mean blood oxygen level, standard deviation of the blood oxygen level, percentage of time blood oxygen level is greater than a threshold value, and mean skewness of the breathing rate

The NCS system sensing tag may be configured to collect dielectric boundary motion on and inside the body, instead of solely relying on surface reflection as in other (e.g., radar-based) methods. The whole-tag motion including user movement and external chest movement may be modulated on the NCS phase, while the relative motion of the internal organ and diaphragm against the tag antenna may be modulated on the NCS amplitude. When the tag is deployed to the chest area, both the respiratory volume and rate can be independently retrieved with minimal inter-sign interferences.

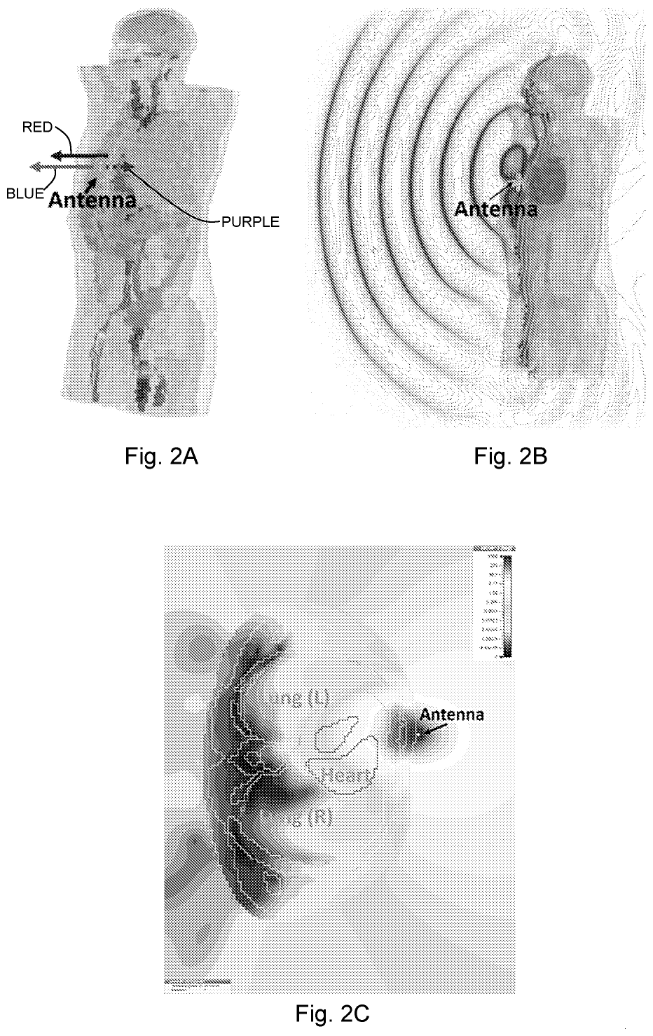

The sensing tag may include dual operating network connectivity. In an active mode, the NCS chest tag will operate with its own battery, sensing and digitally sampling vital signs and providing signals, such as, for example, an alert signal, using one or more techniques including but not limited to audio, visual, radiofrequency (Wi-Fi, Bluetooth, etc.), ultrasound, infrared, and similar techniques (FIG. 2A). A channel scan may be performed to determine the preferred connection method (e.g., having suitable protection and privacy control). When the battery is not properly charged, a dedicated plug-in reader in the home of the user can be implemented to provide additional back-up support (FIG. 2B). In this case, the tag will be operated in the passive mode by the scavenged power like a radio-frequency identification (RFID), and the reader will then broadcast to the wireless network. Such a tag may be considered a semi-active tag. The reader availability can be automatically detected, and the tag will preferentially operate in the passive mode to enable long-term extension of the on-tag battery lifetime. In both active and passive modes, the downlink inquisition signal and uplink-sensing signal can be well separated for minimal self-interference from the harmonic scheme. The NCS sensing range may be, for example, 5-10 cm so that the exact tag placement on the user chest is less critical. In the active mode, the communication range is limited only by the external wireless network. In the passive mode, the reader may be, for example, 10-15 m away, similar to commercial RFID operations.

An exemplary semi-active NCS tag may be provided using a printed-circuit board (PCB) platform with built-in sensing antenna. Packaging may be adapted to different wearable formats and may include a battery pack sustaining more than one day of operation in the active mode. A Wi-Fi module may be embedded for active-mode broadcast. The reader for passive-mode operation may be implemented by a software-defined radio (SDR) platform. The air protocol in the passive mode can follow commercial RFID operation techniques.

The present technique offers the comfort and convenience of the conventional radio-frequency method with a coherent sensing approach that provides: 1) higher sensitivity to retrieve the respiratory dynamics including respiratory volume; 2) network-ready communication in both active and passive modes; 3) the ability to operate in the passive mode with an external reader but without use of a tag battery; and 4) the ability to operate in the active mode without an external reader, which will enable the user to roam free, for example, within the area of an accessible Wi-Fi network. A device according to the present disclosure is based on a near-field coherent sensing (NCS) technique, where the respiratory waveforms can be accurately extracted without skin touch or motion restriction, providing accurate respiratory volumes and rates, as well as airflow velocity in a wireless format such as Wi-Fi (e.g., smart phone). Conventional devices used in polysomnography such as electrocardiogram (ECG), respiratory inductance plethysmography (RIP), and nasal cannula pressure transducers are inconvenient, uncomfortable, and restraining, and thus not suitable for long-term continuous monitoring of opioid users. Radio-frequency (RF) methods such as Doppler far-field backscattering of minute skin motion offer no-touch comfort and convenience, but have non-specific wireless channels that can potentially be interfered by ambient motion covered by the same beam lobe. Moreover, away-from-body nearby readers are required, which negates outdoor use. Also, to improve accuracy for estimating the respiratory volume, the carrier frequency may advantageously be raised to the millimeter-wave level, which not only increases the system cost, but also reduces the operational distance. RF methods that are based on a transmission-line model require good impedance matching of skin electrodes and hence need uncomfortable body surface preparation similar to an ECG.

The term processor is intended to be interpreted broadly. For example, in some embodiments, the processor includes one or more modules and/or components. Each module/component executed by the processor can be any combination of hardware-based module/component (e.g., graphics processing unit (GPU), a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), graphics processing unit (GPU), microprocessor, controller, microcontroller, and/or the like. In a particular example, the processor may include a CPU and a GPU, wherein the GPU is configured as a machine-learning classifier. Other implementations of machine-language classifiers will be apparent in light of the present disclosure and are included in the scope of the present disclosure.

Wearable NCS Device for Respiratory Monitoring with Network Connectivity

In some aspects, the present disclosure provides a wearable monitoring system and method that captures respiratory functions using an NCS technique, where the lung motion can be accurately monitored without skin contact or body motion restriction. The present system provides accurate respiratory measurements such as, for example, breathing rates, respiratory tidal volumes, and/or air flow velocity. As mentioned above, embodiments of the device are capable of detecting respiratory depression, a condition that can precede respiratory arrest and may trigger an alert to the victim and/or designated parties enabling a rapid response. In some embodiments, a dual-mode operation is implemented, for example for both indoor (passive mode) and outdoor use (active mode). In its active mode, the NCS device may broadcast to an established wireless protocol such as, for example, Wi-Fi, Bluetooth, etc. The sensing tag can also operate in the passive mode with a reader. In this mode, it can provide protection even when the user forgets to recharge the battery. In other embodiments, a device may be configured for operation in either passive mode or active mode.

Using NCS methods, RF energy (for example, in the ultra-high frequency (UHF) band (300 MHz-3 GHz)) can be coupled into the body, and detailed dielectric boundary motion in the near-field region of the sensing antenna can be retrieved. Movements of an individual may include, for example, movements related to vital signs—e.g., heartbeat, pulse, breathing, etc. Embodiments of the present method may directly modulate mechanical motion on the body surface or inside the body of the individual onto RF signals in the near-field range. The motion can be modulated onto multiplexed harmonic RF identification (“RFID”) backscattering signals with unique digital identification (“ID”). Embodiments of such a system may utilize a radiation level that is well under the safety standard prescribed by OSHA (Occupational Safety and Health Administration).

The “near field” of an antenna is a region where induction characteristics dominate over radiation characteristics and the relationship between the electric field (E field) and the magnetic field (H field) has not reached the far-field superposition of plane waves. In embodiments of the present disclosure, “near-field” may refer to the close-in region of an antenna where angular field distribution is dependent upon the distance from the antenna. In embodiments, the near-field extends to the region within one wavelength (λ) of the antenna. In other embodiments, the near-field extends to the region within λ/2, λ/3, λ/4, or λ/2π of the antenna, where λ is the operating wavelength of the antenna in the dielectric material(s) under consideration. Other embodiments will be apparent to one having skill in the art with the benefit of the present disclosure.

### Exemplary Embodiment—Monitoring an Opioid User

The following is a non-limiting example of an NCS method for monitoring for respiratory failure (respiratory arrest) of an opioid user. Embodiments of the present method may directly modulate the mechanical respiratory motion the individual onto radio signals, which may be integrated with a unique digital ID. A first radiofrequency (“RF”) sensing signal is provided within a near-field coupling range of a respiratory motion of the individual. The provided first RF sensing signal may be an ID-modulated signal. In some embodiments, the first RF sensing signal is an active radio link. In some embodiments, the first RF sensing signal is a backscattered RFID link. For example, an antenna may emit a beacon or ID-modulated sensing signal in either an active radio link or a backscattering RFID (radio identification) link. The first RF sensing signal will be modulated by the respiratory motion thereby generating a respiratory measurement signal. The method includes detecting the respiratory measurement signal using a first receiver. In some embodiments, the detection may be done at the far field, for example, detecting the respiratory measurement signal transmitted through the body of the individual. In some embodiments, the detection is of a reflected signal, for example, using the near-field antenna.

The first RF sensing signal and the respiratory measurement signal may be matched using a cancellation network. In this way, any first RF sensing signal (i.e., non-modulated first RF sensing signal) detected with the first measurement signal may be reduced. The respiratory motion is measured based on the respiratory measurement signal (which can be with reduced non-modulated first RF sensing signal). In NCS, more energy is directed into the body tissue than previous techniques, so the backscattered signal from internal organs is implicitly amplified. In the case of an ID-modulated wave, multiple mechanical motions may be read simultaneously in a synchronized manner using multiple devices. Multiplexing techniques can be used in passive backscattering or active radio transmission to facilitate simultaneous sensing at multiple points and/or for multiple persons.

The method includes using the measured respiratory motion to detect a respiratory event characteristic of respiratory failure (i.e., respiratory arrest) of an opioid user. In some embodiments, measuring the respiratory motion comprises determining a respiratory rate, determining a respiratory volume, and/or determining an airflow velocity. Respiratory depression may result in a low respiratory rate and/or shallow breathing (i.e, low volume). Non-limiting examples of a low respiratory rate include a rate less than eight breaths per minute or less than 70% of a baseline rate. Examples of shallow breathing include a breath volume less than 250 cc, or less than 70% of a baseline volume. These values are only examples, and other values and/or metrics may be used. The method may further include providing an alert signal if a respiratory event characteristic of a respiratory failure of an opioid user is detected. For example, the alert signal may be one or more of an audible signal, a visual signal, an RF signal, an ultrasound signal, an infrared signal, a haptic signal, and the like.

In some embodiments, the first RF sensing signal is provided from a wireless tag. For example, a downlink signal may be provided to power a wireless tag (received at the tag). The first RF sensing signal is provided from the wireless tag within a near-field coupling range of the first motion to be measured. The first downlink signal may have a frequency—the downlink frequency. The first RF sensing signal may have a frequency—the first RF sensing frequency—that is a harmonic of the downlink frequency. For example, the first RF sensing frequency may be the second harmonic of the downlink frequency. The wireless tag may be, for example, a harmonic radio-frequency identification (RFID) tag or an RFID tag with subcarrier modulation.

In embodiments of the present disclosure, the transmitting and receiving frequencies are coherent. For example, where the tag is a wireless tag, the downlink frequency and the first RF sensing frequency are coherent.

In another aspect, the present disclosure may be embodied as a system for monitoring for respiratory failure (arrest) of an opioid user. The system includes a first signal source for generating a first RF sensing signal. A first Tx antenna is in electrical communication with the first signal source. The first Tx antenna is configured to be disposed within a near-field coupling range of a respiratory motion to be measured. For example, the first Tx antenna may be configured to be disposed within a coupling range of lung motion. In this way, a respiratory measurement signal is generated by the first RF sensing signal being modulated by the respiratory motion. The first RF sensing signal may be an ID-modulated wave. For example, the signal may be an active radio link or a backscattering RFID link.

The system includes a first receiver for detecting the respiratory measurement signal (the first RF sensing signal coupled with (i.e., modulated by) the respiratory motion) and a first Rx antenna in communication with the first receiver. The first receiver and/or the first Rx antenna may be configured to detect the respiratory measurement signal as a transmitted signal— i.e., far-field radiation. The first receiver and/or the first Rx antenna may be configured to detect the respiratory measurement signal as a reflected signal—i.e., antenna reflection. The system may include a filter in communication with the first receiver, wherein the filter is configured to demodulate and filter the respiratory measurement signal to obtain a motion signal. The filter may be, for example, a processor (such as a digital-signal processor (“DSP”)) programmed to sample, demodulate, and/or filter the respiratory measurement signal to derive the respiratory motion signal. The filter may be or may include a bandpass filter configured to filter the respiratory measurement signal using a first frequency range corresponding to the respiratory motion.

The system further includes a processor (e.g., a signal processing circuit, digital signal processor, microprocessor, discrete circuit, etc.) configured to use the measured respiratory motion to detect a respiratory event characteristic of respiratory failure (i.e., respiratory arrest) of an opioid user. In some embodiments, the processor may be configured to determine (i.e., calculate) a respiratory rate, determine a respiratory volume, and/or determine an airflow velocity. The processor may further be configured to provide an alert signal if a respiratory event characteristic of a respiratory failure of an opioid user is detected. For example, the alert signal may be one or more of an audible signal, a visual signal, an RF signal, an ultrasound signal, an infrared signal, a haptic signal, and the like.

In some embodiments, the first RF sensing signal is a frequency-doubled downlink frequency. A Tx frequency doubler which doubles a downlink frequency before transmission via Tx antenna.

The system may include a second signal source for generating a second sensing signal. A second Tx antenna may be in electrical communication with the second signal source. The second Tx antenna is configured to be disposed within a near-field coupling range of a second motion to be measured. For example, the second Tx antenna may be configured to be disposed within a coupling range of a heart motion, a pulse, a respiration motion, a bowel motion, an eye motion, etc. In this way, a second measurement signal is generated by the second sensing signal being modulated by the second motion. The second sensing signal may be an ID-modulated wave. For example, the signal may be an active radio link or a backscattering RFID link.

The system may further include a second receiver for detecting the second measurement signal (the second sensing signal coupled with (i.e., modulated by) the second motion) and a second Rx antenna in communication with the second receiver. The second receiver and/or second first Rx antenna may be configured to detect the second measurement signal as a transmitted signal—i.e., far-field radiation. The second receiver and/or the second Rx antenna may be configured to detect the second measurement signal as a reflected signal—i.e., antenna reflection.

The NCS system sensing tag may be configured to collect dielectric boundary motion on and inside the body, instead of solely relying on surface reflection as in other (e.g., radar-based) methods. The whole-tag motion including user movement and external chest movement may be modulated on the NCS phase, while the relative motion of the internal organ and diaphragm against the tag antenna may be modulated on the NCS amplitude. When the tag is deployed to the chest area, both the respiratory volume and rate can be independently retrieved with minimal inter-sign interferences.

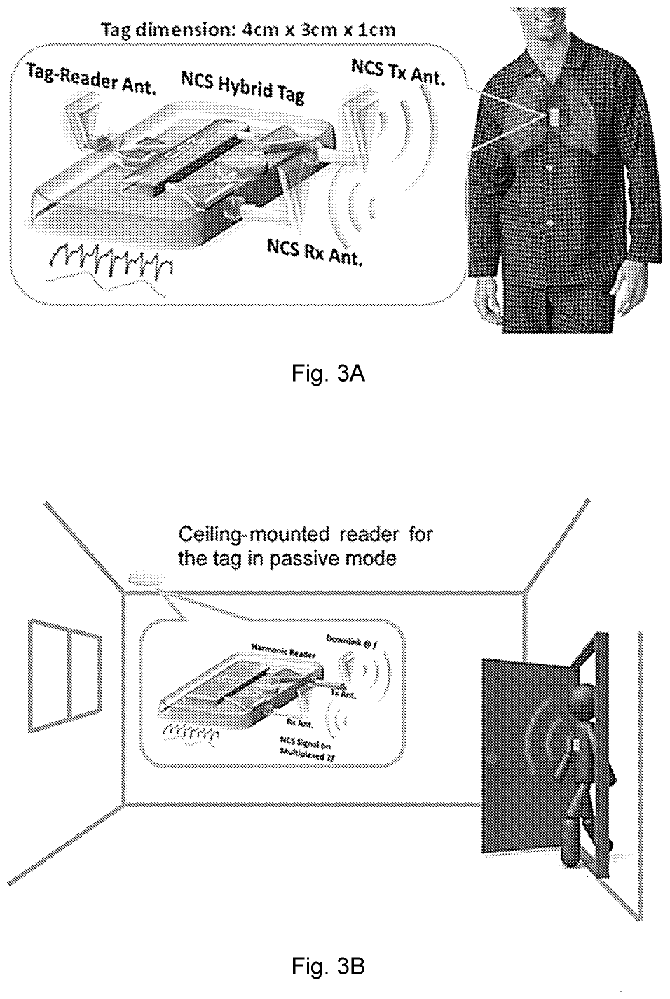

The sensing tag may include dual operating network connectivity. In an active mode, the NCS chest tag will operate with its own battery, sensing and digitally sampling vital signs and providing signals, such as, for example, an alert signal, using one or more techniques including but not limited to audio, visual, radiofrequency (Wi-Fi, Bluetooth, etc.), ultrasound, infrared, and similar techniques (FIG. 3A). A channel scan may be performed to determine the preferred connection method (e.g., having suitable protection and privacy control). When the battery is not properly charged, a dedicated plug-in reader in the home of the user can be implemented to provide additional back-up support (FIG. 3B). In this case, the tag will be operated in the passive mode by the scavenged power like a radio-frequency identification (RFID), and the reader will then broadcast to the wireless network. Such a tag may be considered a semi-active tag. The reader availability can be automatically detected, and the tag will preferentially operate in the passive mode to enable long-term extension of the on-tag battery lifetime. In both active and passive modes, the downlink inquisition signal and uplink-sensing signal can be well separated for minimal self-interference from the harmonic scheme. The NCS sensing range is typically at 5-10 cm so that the exact tag placement on the user chest is less critical. In the active mode, the communication range is limited only by the external wireless network. In the passive mode, the reader can be 10-15 m away, similar to commercial RFID operations.

An exemplary semi-active NCS tag may be provided using a printed-circuit board (PCB) platform with built-in sensing antenna. Packaging may be adapted to different wearable formats and may include a battery pack sustaining more than one day of operation in the active mode. A Wi-Fi module may be embedded for active-mode broadcast. The reader for passive-mode operation may be implemented by a software-defined radio (SDR) platform. The air protocol in the passive mode can follow commercial RFID operation techniques.

### Respiratory Events

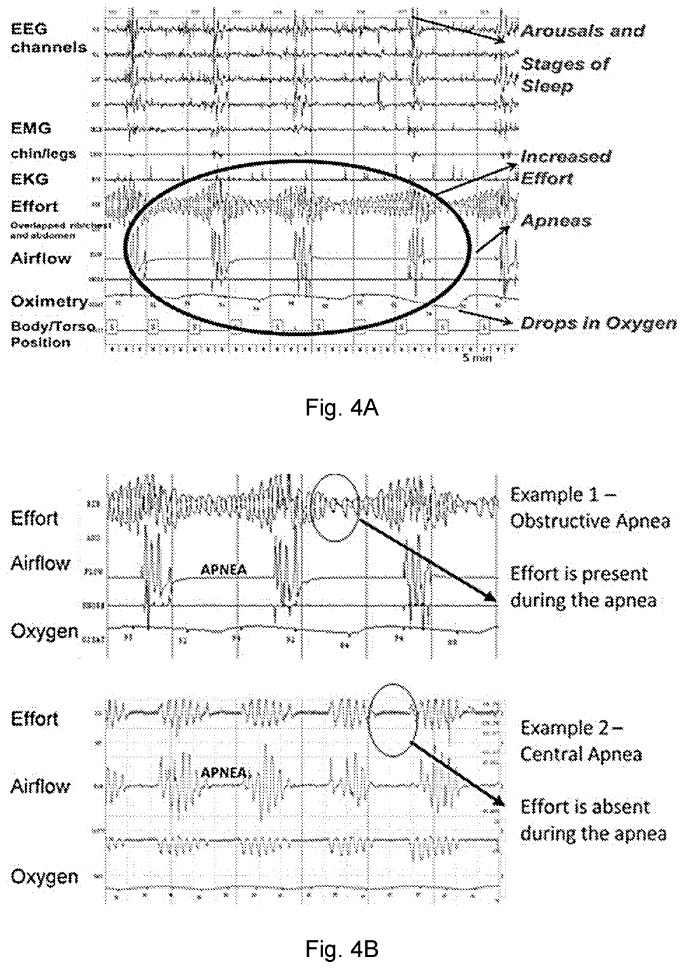

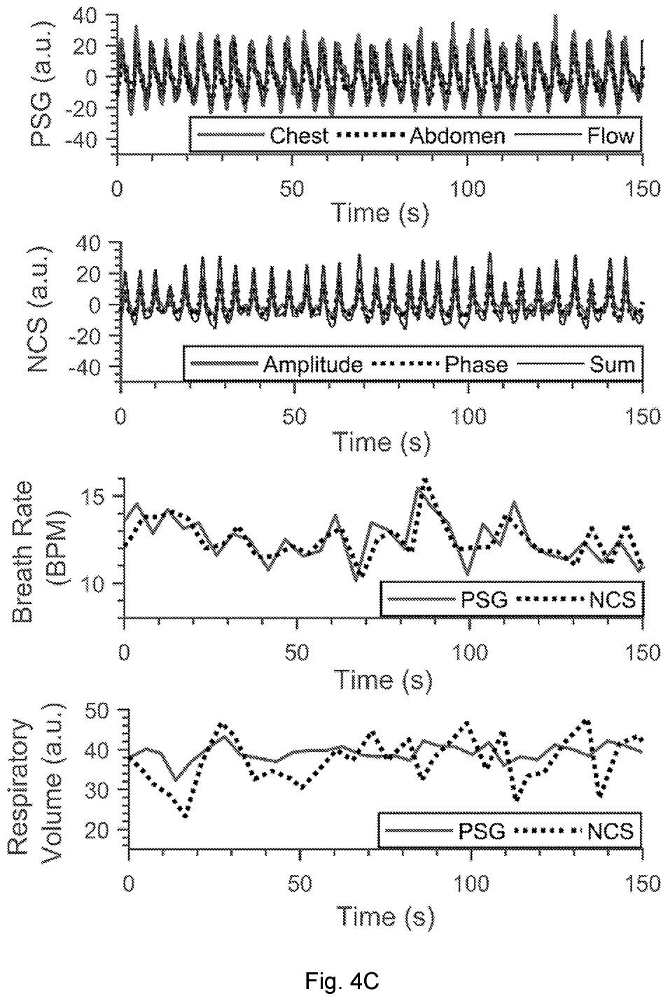

Physiological signals and respiratory events, including hypoventilation, hypopneas, central and obstructive apneas, as demonstrated in FIGS. 4A-4C, can be used as a model of potential respiratory abnormalities to validate the present NCS method and to develop a respiratory event signature.

The airflow tracing can be recorded and interpreted by the evaluation of its shape, amplitude, and frequency. Respiratory events may comprise apneas (complete or over 90% reduction in airflow—for example, lasting at least 10 seconds) and/or hypopneas (reduction in amplitude of at least 50%). These are only exemplary values, and such events may be measured differently according to a particular application (e.g., apneas may be measured as lasting at least 10 second, 20 second, 30 seconds, or otherwise). Events may be categorized based on their potential etiology to determine if they are due to a mechanical restriction in airflow (obstructive) or are centrally-mediated (central) (FIGS. 4A-4C). In applications for monitoring opioid users, lethal opioid overdoses have been reported as with central (lack of CNS input) and/or obstructive (due to chest wall rigidity) events.

Identification of respiratory events may be performed using known approaches (see, for example, J. Hosselet et al., “Classification of sleep-disordered breathing,” Am J Respir Crit Care Med, 2001 February, 163(2):398-405; and B. Dubrovsky et al., “Polysomnographic investigation of sleep and respiratory parameters in women with temporomandibular pain disorder,” J Clin Sleep Med, 2014 Feb. 15, 10(2):195-201), and may include the addition of apnea (pauses in breathing, for example, a 30 second pause) identification, which may be used to verify the sensitivity of the system and categorized as a “critical event” requiring increased sensitivity. In some embodiments, a combination of signals including, for example, other cardiovascular signals, oximetry, and/or motion can be used to improve the validity of the NCS system, including techniques to assist in the validation of pre-critical events including 10 second apneas, hypopneas, and/or episodes of hypoventilation.

### Demonstration of Semi-Active NCS Tags for System Models

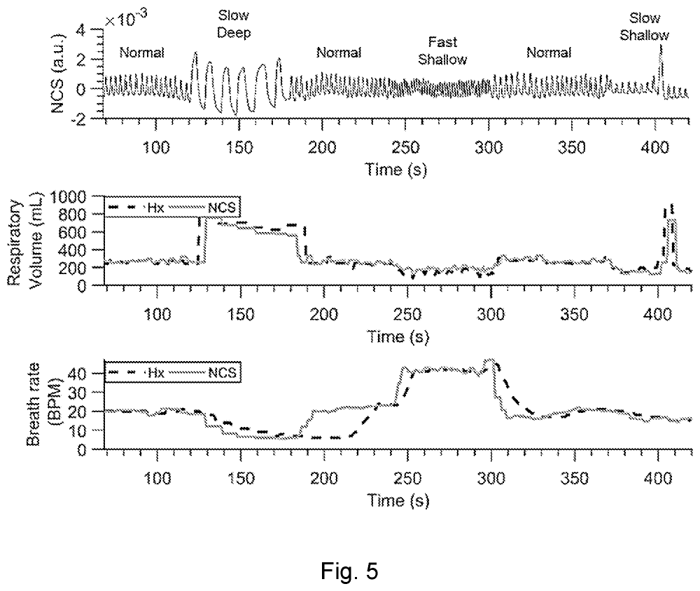

In a non-limiting test embodiment to demonstrate the preliminary feasibility of the present NCS system to monitor the respiratory efforts, autonomous breathing in various patterns was recorded by both an exemplary NCS system and the respiratory inductive plethysmography (RIP) sensors of the commercial Hexoskin (Hx) shirt in FIG. 5. While the respiratory rate was automatically detected right after setup, initial calibration specific to the user was still performed for respiratory volume extraction. During breathing exercises, extraction of both tidal volumes and respiratory rates matched well between NCS and Hexoskin. Accurate and detailed recording of transient changes in respiratory tidal volume, extracted from peak inhalation and exhalation, in addition to changes in respiratory rate, may be used for an early warning of a critical event preceding respiratory arrest and will be sufficient to provide low false negative readings.

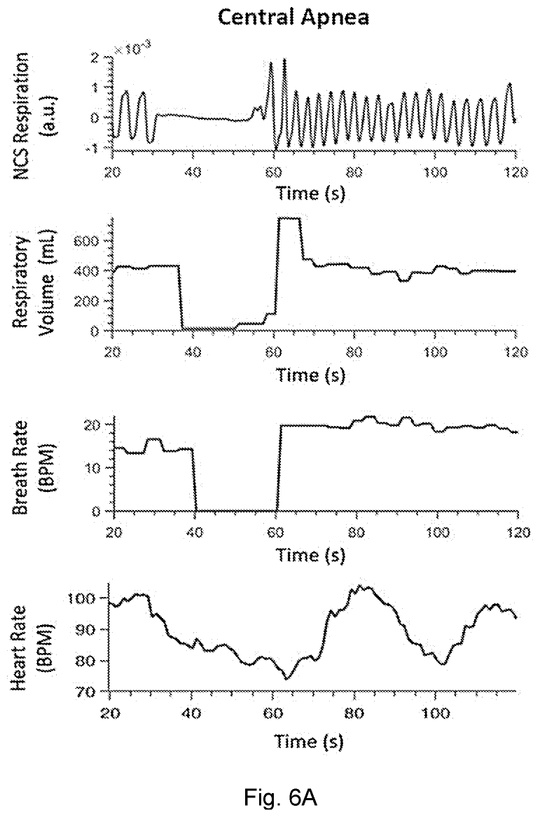

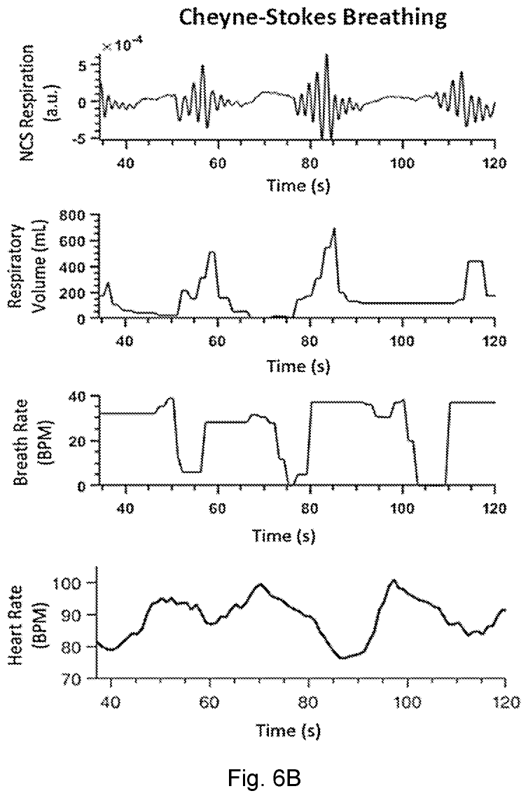

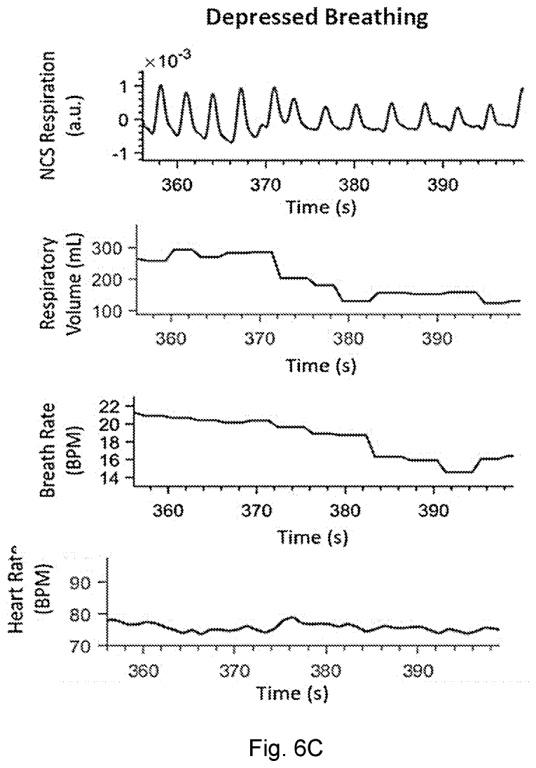

For example, without the respiratory volume information, in obstructed apnea, out-of-phase thorax and abdomen movement may be wrongly identified as false respiratory rates while no air is exchanged with the lung. For preliminary proof-of-concept illustration, further studies of respiratory disorders were conducted including central apnea, Cheyne-Stokes breathing, and depressed respiration that were simulated by actors where the signature respiratory volumes and breath rates were clearly observed (FIGS. 6A-6C). The synchronous cardiogram during these exercises was extracted as well, to evaluate the use of heartbeat information to improve detection accuracy with extended warning time.

NCS measures the dielectric boundary movement within the near field region to be represented by RF antenna characteristics; RIP measures the inductance change due to chest belt expansion. In this perspective, NCS is a more direct and sensitive measurement for the mechanical motion of pulmonary functions than ECG and RIP. Therefore, direct calibration of NCS against ECG, RIP and flow meters can be misleading due to the errors and variations that exist in each method. To provide a more reliable design and pin down the interference and variation sources, known ground truth of respiratory actions can be used. For example, a phantom model can be used, where the respiratory rate and volume can be accurately repeated without human variations.

Although NCS can reasonably isolate the common-mode motion (i.e., the sensing antenna and the motion source move together) and differential-mode motion (i.e., the motion between the sensing antenna and motion source) by the demodulated phase and magnitude in the standard quadrature scheme, the vital-sign signal can still be interfered with by large body movements when the characteristic spectral components of body motion are close to those of the vital signs. The NCS tag in the active mode may be less vulnerable to body motion as the sensing is entirely completed and digitized on the tag. Subsequent digital communication can use conventional error correction codes to negate any motion interference that affects the indoor channel. When the tag is in the passive mode, it can be overwhelmed by large common-mode motion and ambient motion. Further mitigation of user motion interference may be performed by advanced signal processing and garment integration.

### Heartbeat Signal

Use of a cardio waveform can play a role in providing better accuracy and provide an earlier warning preceding a respiratory arrest. The present NCS system and method may be used to obtain a clear heartbeat waveform by the chest tag simultaneously with little or no additional setup or cost. For example, the respiratory motion signal may include heart rate information, which may be at a higher frequency than the respiration information. As such, the respiratory measurement signal can be filtered to distinguish the cardiac information. In some embodiments, the heartbeat is a reliable signal that can be used for validation of the tag functioning. For example, if the user places the tag away from the chest or misuses the tag intentionally or unintentionally, then both cardiopulmonary waveforms (heartbeat and respiratory motion) will be lost suddenly and the present system may be configured to send an appropriate warning message to the recipient(s) (e.g., different from a respiratory arrest alert message). This may reduce the false alarm rate significantly. In some embodiments, machine-learning techniques may be used to detect the presence of cardio waveform signatures preceding apnea, hypopnea, and/or respiratory arrest.

### Second Measurement Signal

The NCS sensing approach is based on the near-field coupling of RF (e.g., UHF) waves with the nearby dielectric boundary motion. The dielectric composition in the near-field region of an antenna will modulate its characteristics. For a single antenna, this change can be measured from the antenna reflection parameter S11. For an antenna pair, this can be derived from, for example, the cross-coupling S21. As the transmitter (Tx) and receiver (Rx) signal chains are better isolated in the S21 measurements with less self-interference and higher signal-to-noise ratio (SNR), embodiments of an NCS sensor may use an antenna pair, which can be placed at a region of interest, where the intended surface and internal boundary motion can be retrieved after baseband demodulation. Notice that UHF has reasonable penetration into dielectrics in the near-field, and thus the internal dielectric motion during breathing and heartbeat can be locally modulated onto the specific antenna pair.

In an experimental embodiment, two NCS sensors were placed on the chests of study participants, one below the xiphoid process close to the diaphragm (abdominal sensor), and another near the heart (thoracic sensor). These sensors measure the abdomen and thorax respiratory motion as well as the heartbeat, by effectively capturing the geometrical changes in these organs along with other associated muscles. The acquired NCS signals were processed to estimate respiratory volume (RV), breathing rate (BR), and heart rate (HR) statistics. Heartbeat was also modulated clearly on the NCS thorax waveform and could be filtered to remove respiratory motion.

### Paradoxical Abdomen-Thorax (PAT) Motion

Certain respiratory conditions may be detected, monitored, and/or predicted using an embodiment of the present disclosure having two NCS sensors—an abdominal sensor and a thoracic sensor. Such conditions include, for example, asthma, chronic obstructive pulmonary disease (COPD), and obstructive sleep apnea. In such cases, increased breathing effort can result in a difference in phase of the measurement signals from each sensor. For example, an abdominal motion may precede a thoracic motion or may have a difference in amplitude and/or direction of movement from an associated thoracic motion.

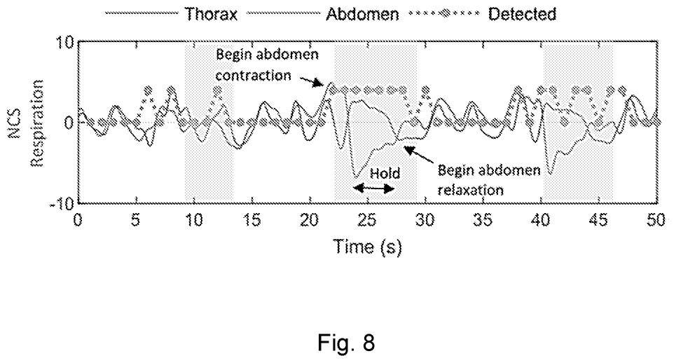

To test the separate thorax and abdomen motion, participants in a study were asked to perform an isovolumetric abdomen exercise while holding breath. With no airflow, the inward abdomen contraction results in outward motion of the thorax, as the total lung volume is conserved, simulating paradoxical abdomen-thorax motion similar to obstructive sleep apnea (OSA) with complete closure of the airway. The slope-product of thorax and abdomen respiration waveforms was used to detect paradoxical motion in NCS waveforms, as shown in FIG. 8, where three instances of isovolumetric maneuver are successfully detected by the sensors. The intended paradoxical motion windows are marked by shaded areas and detected instances are shown by the positive value of dotted lines. Timing of abdomen contraction, hold, and relaxation is denoted during the second cycle of the NCS waveform. NCS was able to detect all three instances of paradoxical abdomen-thorax motion. The expected paradoxical behavior was observed in several participants. Overall, the experimental algorithm was designed to be able to detect slight paradoxical motion, resulting in the sensor performance shown in Table 1.

TABLE 1

| | Detected | Missed |
|---|---|---|
| Breath Hold (BH) (n = 80) | 77 | 3 |
| PAT (n = 58) | 48 | 10 |

### Respiratory Event Prediction

A near-field coherent sensing device was integrated into the furniture of two bedrooms at the Weill Cornell Center for Sleep Medicine. The sensors were used to capture the respiratory signatures of patients during concomitant overnight monitoring. Respiratory rate, tidal volume, and heart rate parameters during wakefulness and sleep.

A total of 30 participants undergoing overnight polysomnography were enrolled in this study and completed participation. Real-time data was acquired including respiratory flow/amplitude, respiratory rate, and heart rate for all participants using the gold-standard polysomnography monitoring and the presently-disclosed NCS device. Data was collected during sleep, and the data was analyzed in time epochs to determine if respiratory events could be predicted in the 1-2 epochs before the event. Time epochs may be selected according to the application, the data, and/or other considerations. For example, epochs may range from 5 second to 5 minutes or longer. In some embodiments, the epoch is a value selected from between 15 seconds and 45 seconds. In some embodiments, the epoch may be 10, 20, 30, 40, 50, or 60 seconds.

For the experiment, sixteen respiratory features were considered: mean breathing rate (“mean(BR)”), breathing rate standard deviation (“std(br)”), breathing rate coefficient of variation (COV)(“covBR”), mean peak-to-peak (“mean(pp)”), peak-to-peak standard deviation (“std(pp)”), peak-to-peak COV (“covPP”), mean inhalation time (“mean(in)”), inhalation time standard deviation (“std(in)”), mean exhalation time (“mean(ex)”), exhalation time standard deviation (“std(ex)”), skewness, kurtosis, entropy, Power Ratio (power spectral density in [0.05, 0.5] Hz/power spectral density in all frequencies) (“per power” or “psdRatio”), breathing cycle number (total number of detected breathing cycles in the specific epoch), and time duration that no peak is detected (“void t”).

For the experiment, four blood oxygen (SpO₂) features were considered: mean SpO₂ (“mean(spo₂)”), SpO₂ standard deviation (“std(spo₂)”), percent of time during which SpO₂ was less than a threshold value of 92%, and minimum SpO₂.

The ability to predict a respiratory event (e.g., an apnea) in 1-2 epochs before the occurrence was evaluated. If a current epoch was detected as disordered (e.g., current epoch is classified as snore, RERA, hypopnea, OSA, mixed apnea, or CSA), then such current epoch was disregarded for prediction.

Each epoch was labeled based on scores provided in sleep annotations for the participants. Scores were numbered as follows:

| Score | Description |
|---|---|
| 0 | No annotation |
| 1 | sleep stage N1 |
| 2 | sleep stage N2 |
| 3 | sleep stage “wake” |
| 4 | sleep stage REM |
| 5 | snore |
| 6 | arousal |
| 7 | RERA |
| 8 | O2 desaturation |
| 9 | Hypopnea |
| 10 | Obstructive Apnea |
| 11 | Mixed Apnea |
| 12 | Central Apnea |

Labels were determined according to the scores where an epoch was labeled with a disorder if the time points with an apnea score were greater than 0.4× epoch duration. If the duration of an epoch with no annotation (score=0) was greater than 0.5× the epoch duration, the epoch was deleted.

| Label | Description |
|---|---|
| 0 | No disorder |
| 1 | Snore |
| 2 | RERA + arousal |
| 3 | Hypopnea + O₂ desaturation |
| 4 | OSA |
| 5 | Mixed Apnea |
| 6 | CSA |

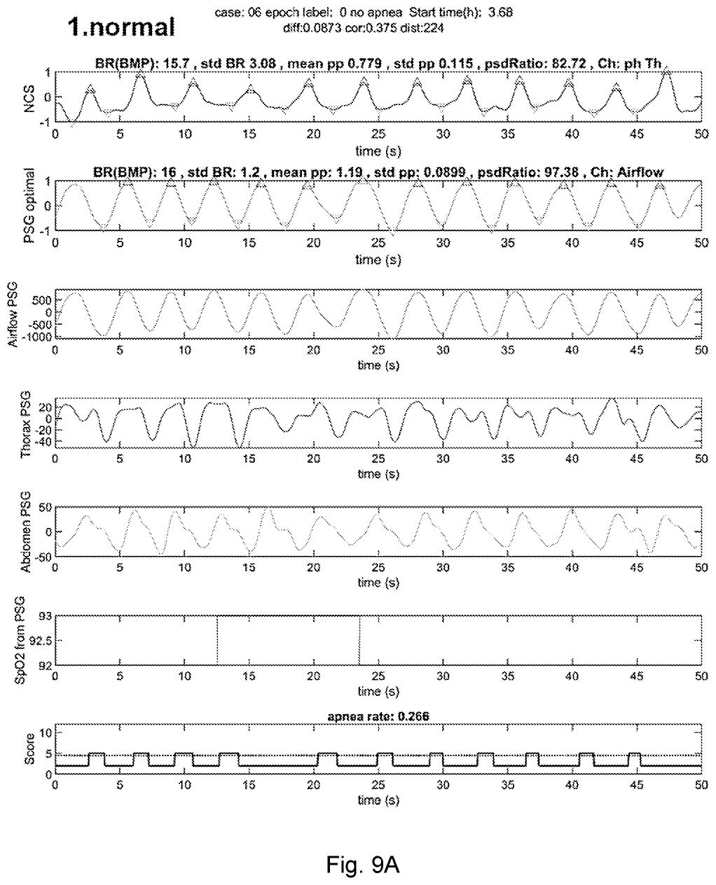

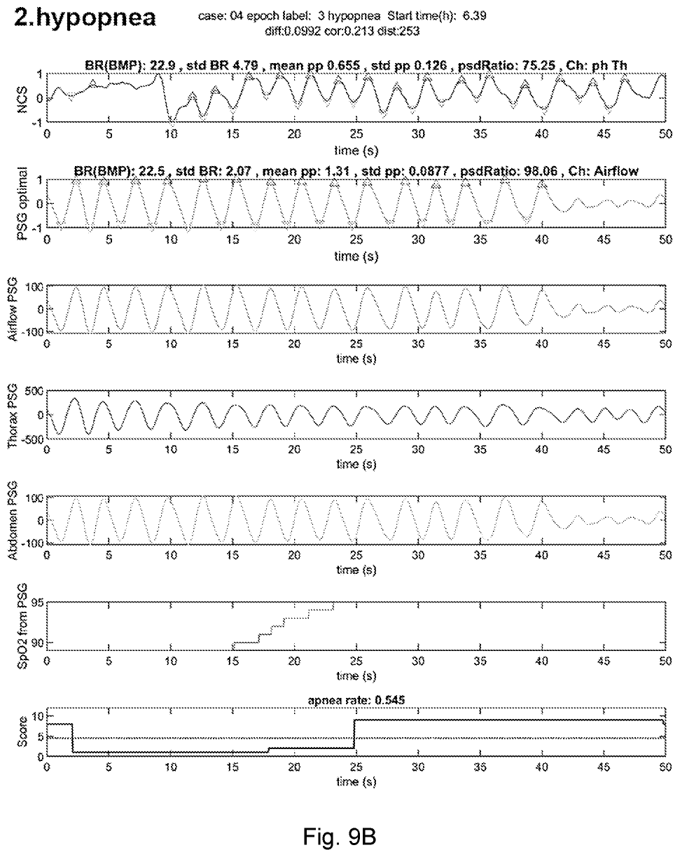

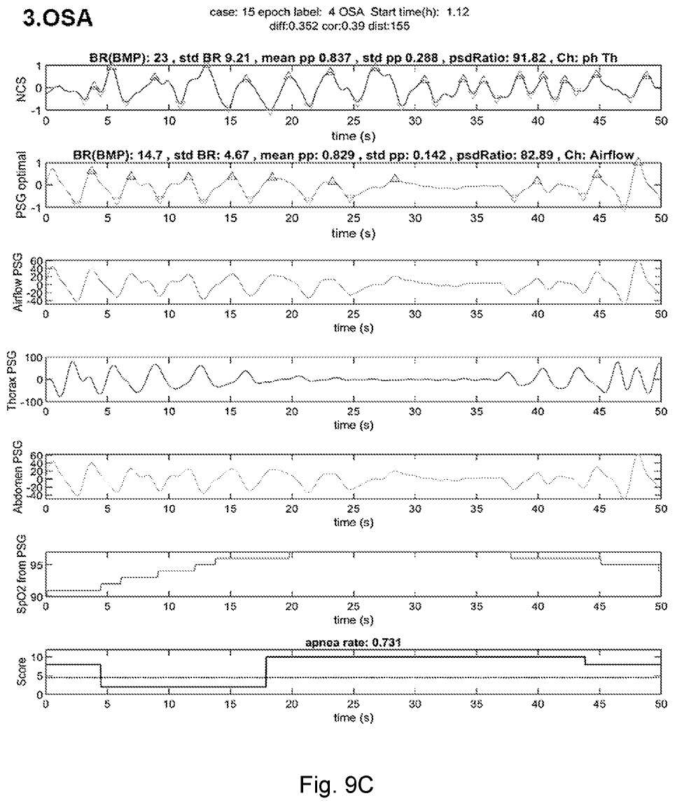

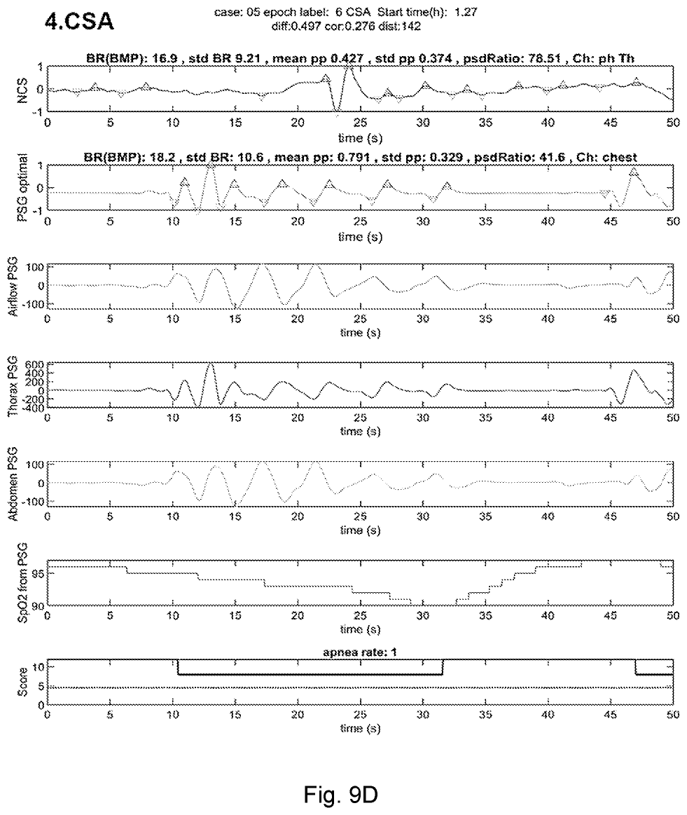

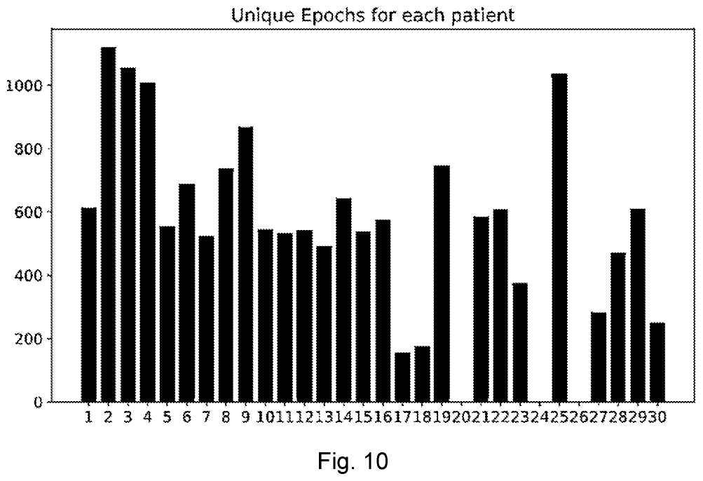

FIGS. 9A-9D show example waveforms captured using NCS (top most graph in each figure), using PSG, and scored data (bottom most graph in each figure). FIG. 9A shows a normal epoch (label 0), FIG. 9B shows an epoch of label 3 (Hypopnea+O₂ desaturation); FIG. 9C shows an epoch of label 4 (Obstructive Sleep Apnea); and FIG. 9D shows an epoch of label 6 (Central Sleep Apnea).

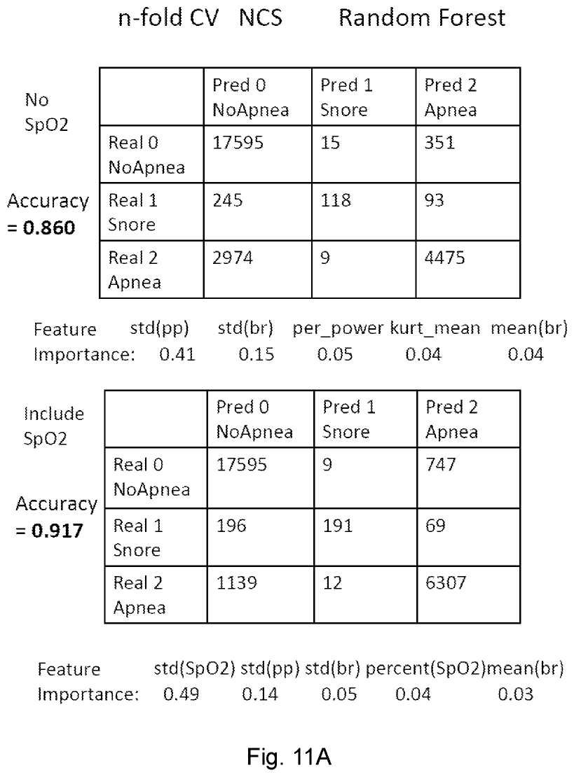

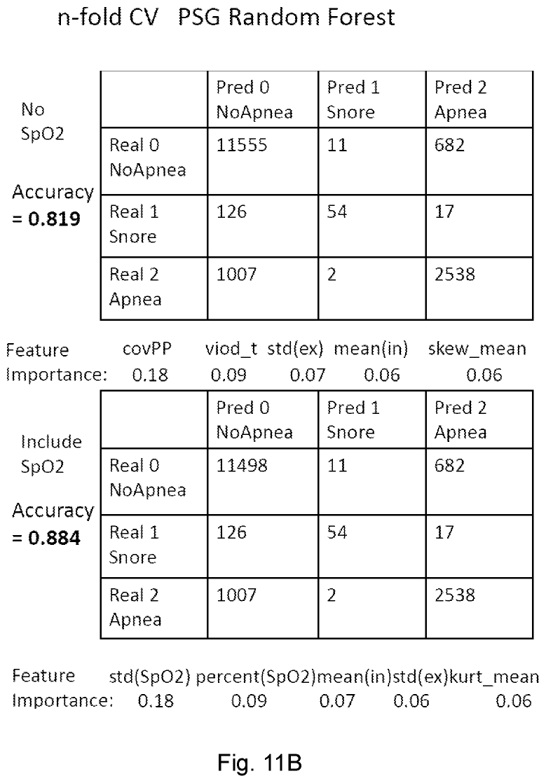

FIGS. 11A and 11B show cross validation results of a random forest classifier using NCS data (FIG. 11A) and PSG data (FIG. 11B) to classify between “NoApnea” (no apnea and RERA), “Snore,” and “Apnea” (OSA, CSA, mixed apnea, and hypopnea).

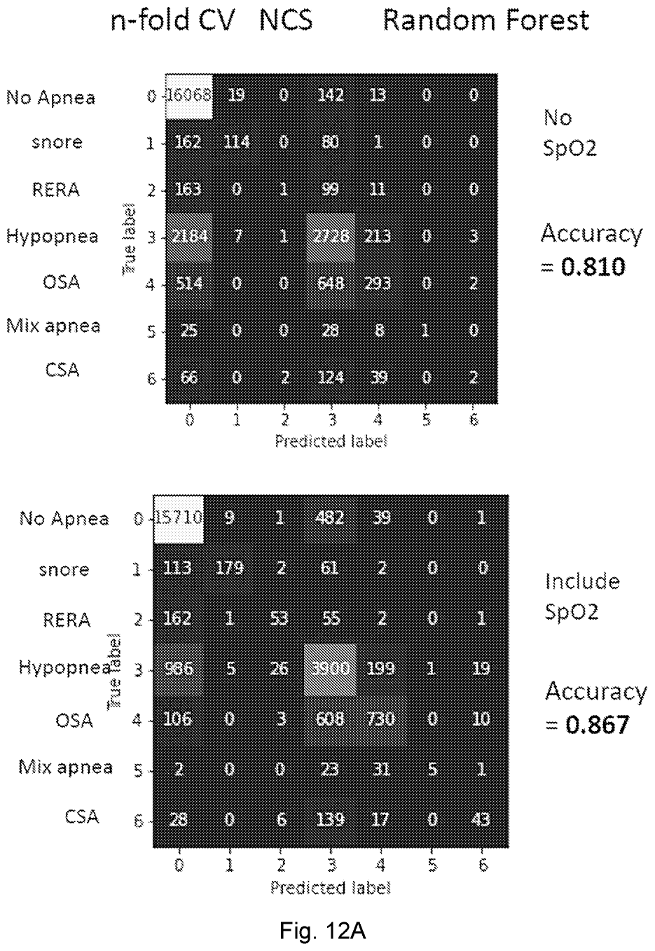

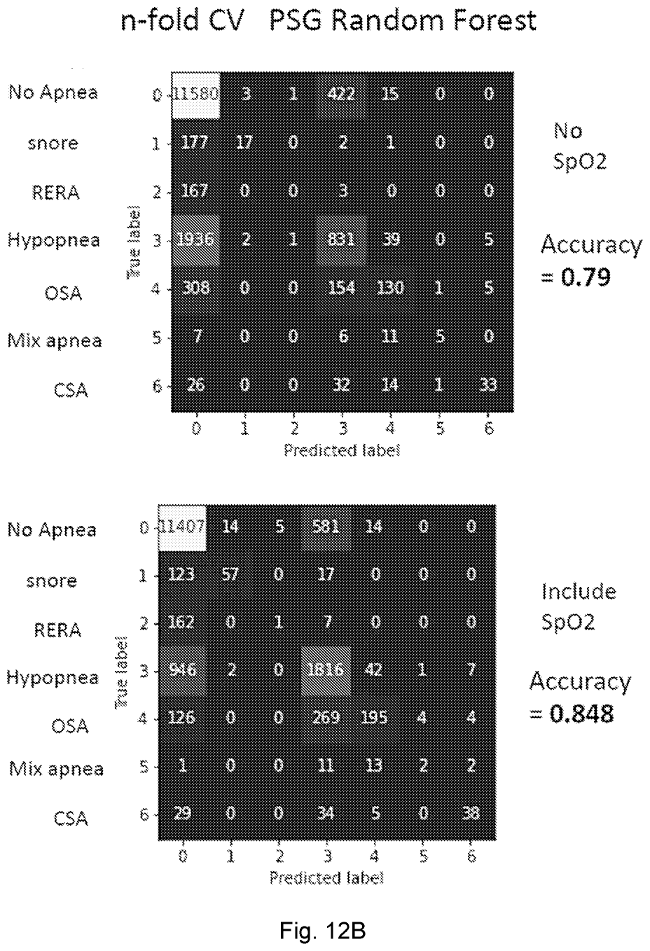

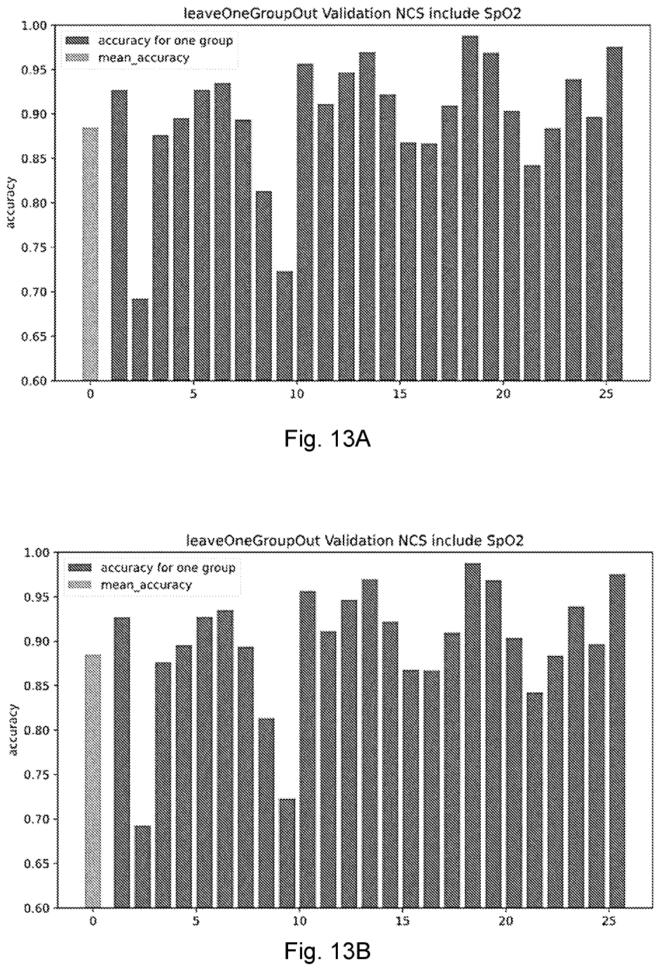

FIGS. 12A and 12B show cross validation results of a random forest classifier using NCS data (FIG. 12A) and PSG data (FIG. 12B) to classify each disorder separately. In each figure, the top results were for a classifier which did not use SpO₂ features, and the bottom results were for a classifier using SpO₂ features.

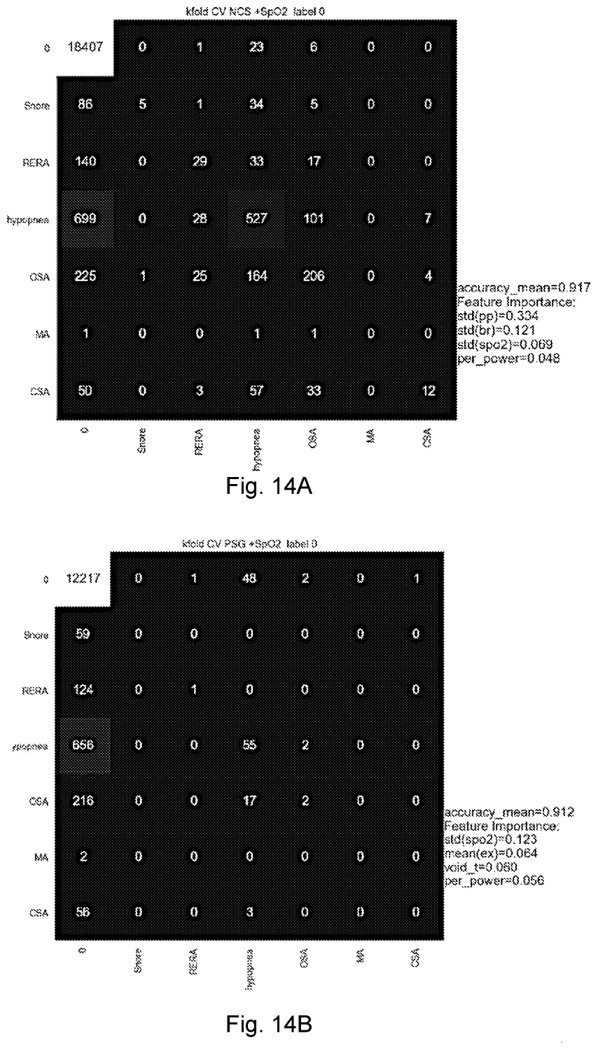

FIGS. 14A-17B show results related to predicting disordered events within 1-2 epochs before the occurrence. For this study, the epoch duration was 40 seconds, and the prediction time was therefore 0-80 seconds before occurrence.

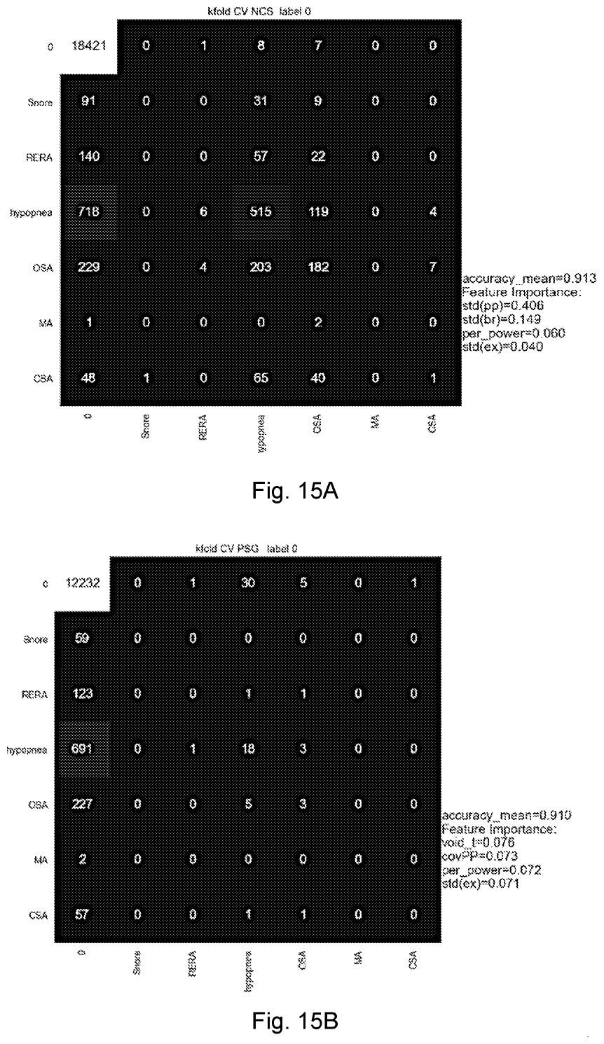

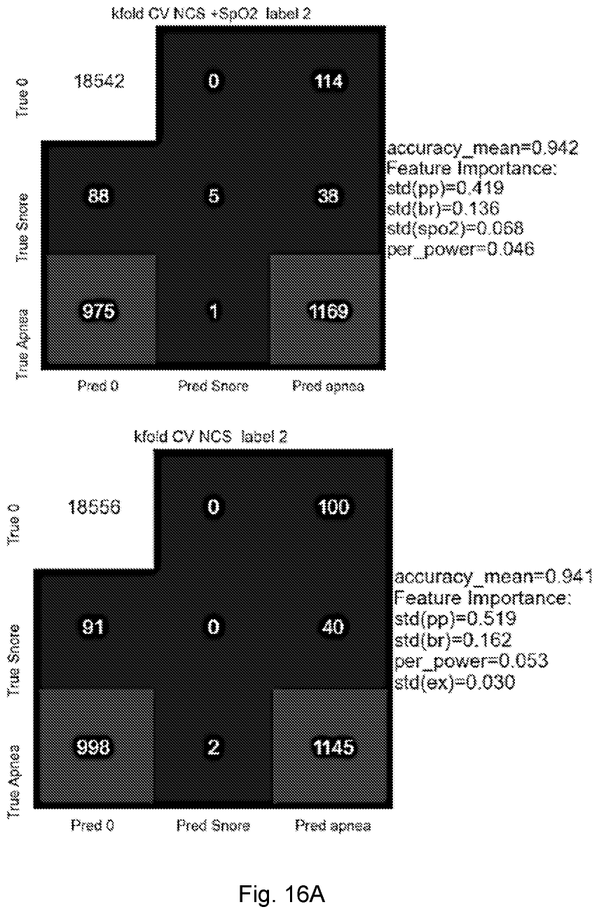

FIGS. 14A-14B show the results of predicting a disorder before it happens in 1-2 epochs using NCS (FIG. 14A) and PSG (FIG. 14B), and including SpO₂ features. FIGS. 15A-15B show the results of predicting a disorder before it happens in 1-2 epochs using NCS (FIG. 15A) and PSG (FIG. 15B), excluding SpO₂ features. In FIGS. 14A-15B, each disorder was classified separately.

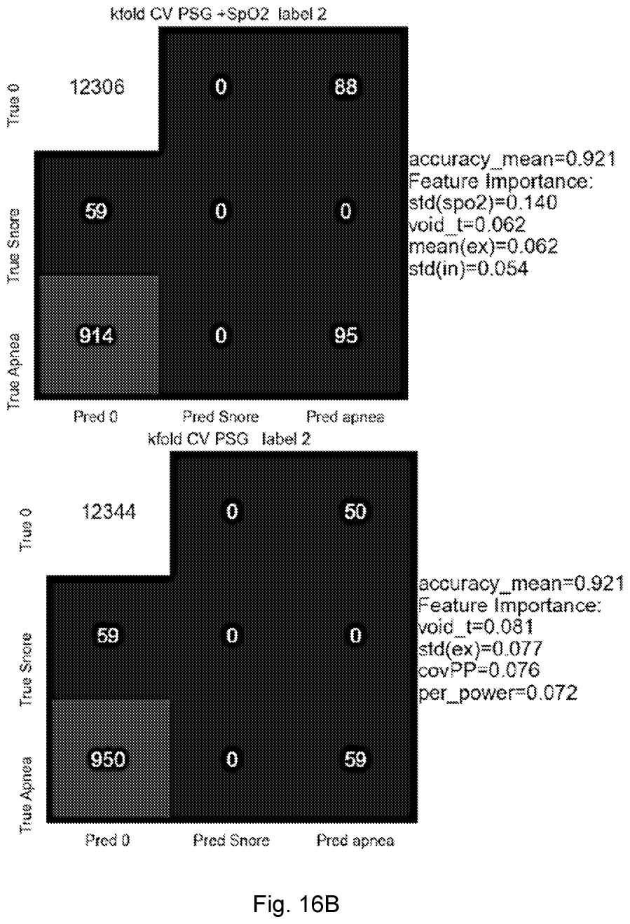

FIG. 16A shows the results of predicting a disorder before it happens in 1-2 epochs using NCS, where the top results show a classifier which included SpO₂ features, and the classifier of the bottom results excluded SpO₂ features. The disorders were classified as either “0” (no apnea and RERA), “Snore,” or “Apnea” (OSA, CSA, mixed apnea, and hypopnea). FIG. 16B shows charts similar to those of FIG. 16A, but using PSG data rather than NCS.

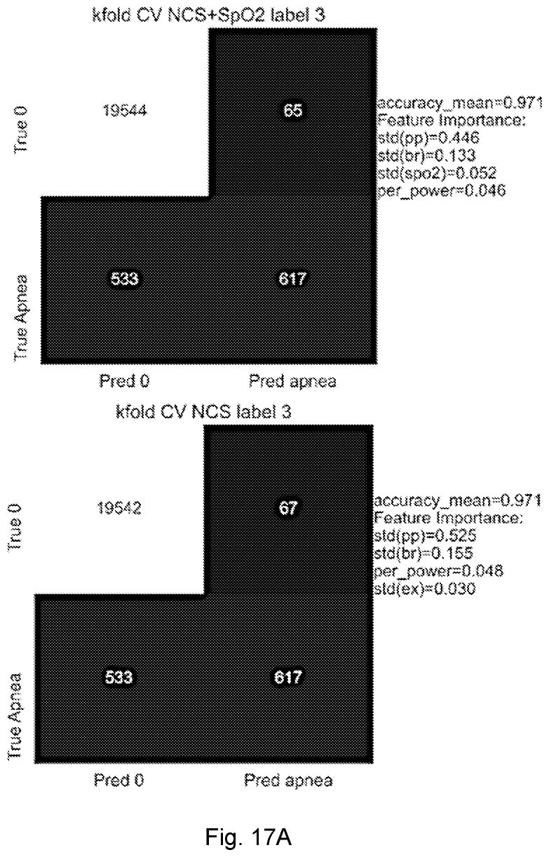

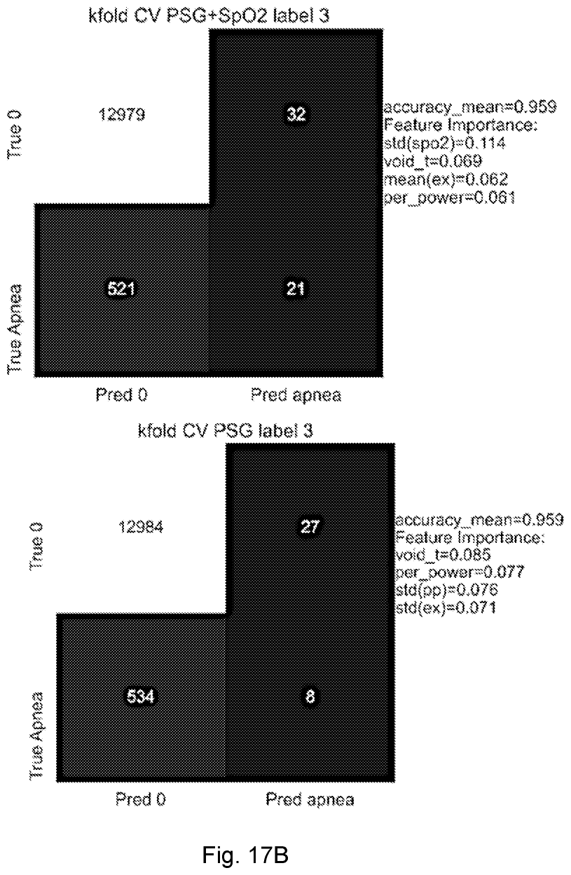

The results of FIGS. 16A-16B indicate that “snore” was not accurately predicted. The classifier results without snore are shown in FIGS. 17A-17B. FIG. 17A shows the results of predicting a disorder before it happens in 1 epoch using NCS, where the top results show a classifier which included SpO₂ features, and the classifier of the bottom results excluded SpO₂ features. The disorders were classified as either “0” (no apnea and RERA) or “Apnea” (OSA, CSA, mixed apnea, and hypopnea). FIG. 17B shows charts similar to those of FIG. 17A, but using PSG data rather than NCS.

### Sleep Stage Characterization

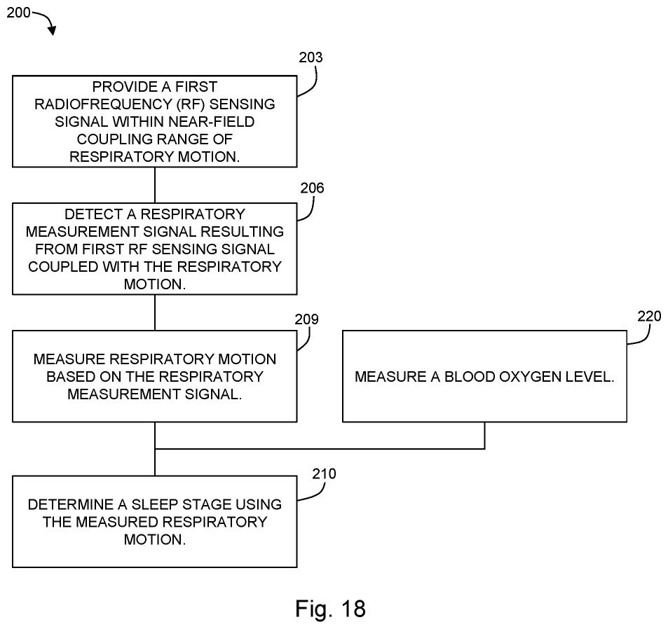

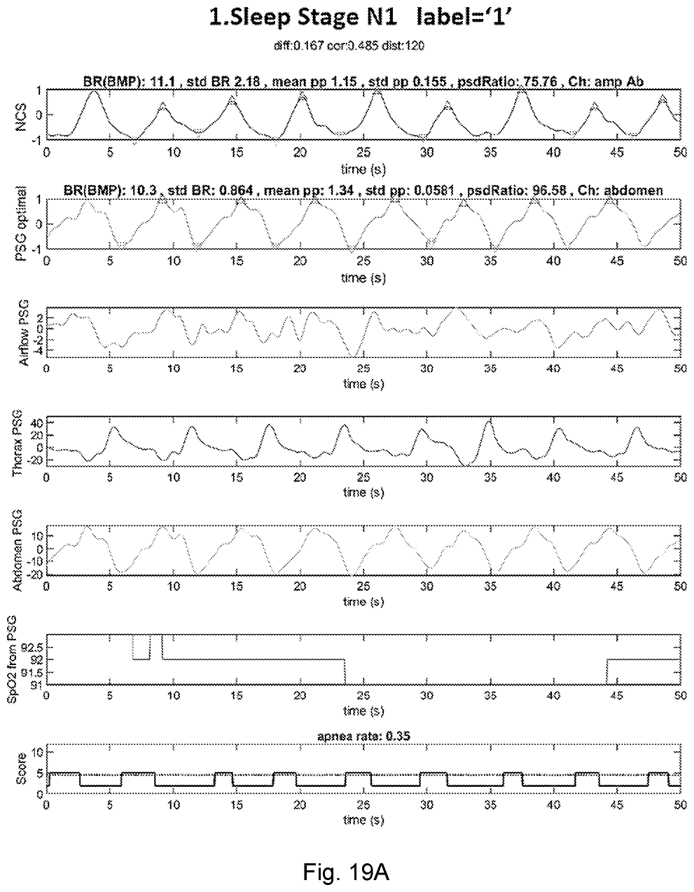

With reference to FIG. 18, the present disclosure may be embodied as a method 200 for characterizing sleep of an individual. The method 200 includes providing 203 a first radiofrequency (“RF”) sensing signal within a near-field coupling range of a respiratory motion to be measured to generate a respiratory measurement signal as the first RF sensing signal modulated by the respiratory motion. For example, a first antenna may be used to transmit a first RF sensing signal. A respiratory measurement signal is detected 206, where the respiratory measurement signal comprises the first RF sensing signal coupled with the respiratory motion of the individual. Continuing the previous example, a receiver may use the first antenna to receive the respiratory measurement signal as a reflected signal. In another example, a receiver may use an antenna located in the far field to detect the respiratory measurement signal as the coupled first RF sensing signal transmitted through the respiratory motion.

The provided first RF sensing signal may be an ID-modulated signal. In some embodiments, the first RF sensing signal is an active radio link. In some embodiments, the first RF sensing signal is a backscattered RFID link. For example, an antenna may emit a beacon or ID-modulated sensing signal in either an active radio link or a backscattering RFID (radio identification) link. The first RF sensing signal and the respiratory measurement signal may be matched using a cancellation network. In this way, any first RF sensing signal (i.e., non-modulated first RF sensing signal) detected with the first measurement signal may be reduced.

The respiratory motion is measured 209 based on the respiratory measurement signal. One or more parameters may be measured 209. For example, one or more of a breathing rate of the individual a peak-to-peak amplitude of the respiratory motion, an inhalation time, an exhalation time, a time duration during which no peak is detected (void time), a Power Ratio (further defined below), respiratory volume, and airflow (e.g., velocity). Such measurements may each include one or more of a mean, standard deviation, coefficient of variation (COV), skewness, kurtosis, and/or entropy of the measurement (e.g., the measurement over a period of time—the “measurement period”). A “Power Ratio” may be determined as a power spectral density of the respiratory measurement signal over a relevant bandwidth, divided by the power spectral density of the respiratory measurement signal over the entire measured bandwidth. An exemplary relevant bandwidth may be an expected range for respiration—for example, 0.05 Hz to 0.5 Hz.

The method 200 includes determining 210 a sleep stage of the individual using a machine learning classifier. The machine learning classifier may be trained to determine the sleep stage using, for example, one or more parameters measured from the respiratory measurement signal (for example, as described above). Such parameters may include mean breathing rate, breathing rate standard deviation, breathing rate coefficient of variation (COV), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, Power Ratio, breathing cycle number (total number of detected breathing cycles in the specific epoch), and time duration that no peak is detected.

In some embodiments, the method 200 includes measuring 220 a blood oxygen level of the individual over the measurement period. The machine learning classifier may be trained using one or more of: mean blood oxygen level, standard deviation of the blood oxygen level, percentage of time blood oxygen level is greater than a threshold value (e.g., a predetermined threshold value), and mean skewness of the breathing rate. The steps of the method may repeated such that the sleep stage of the individual is determined periodically during the measurement period. The method may include determining a wellness of the individual based on the periodically determined sleep stage. Relationships between sleep stages and wellness are known and may be implemented as part of the method. The wellness may be displayed to the individual and/or a caregiver using, for example, a software application (e.g., a smartphone app, etc.)

The disclosure may be embodied as a system for characterizing sleep of an individual. Such a system may be similar to the system 10 depicted in FIG. 1, where the processor 20 includes a classifier trained to determine a sleep stage of an individual as described above.

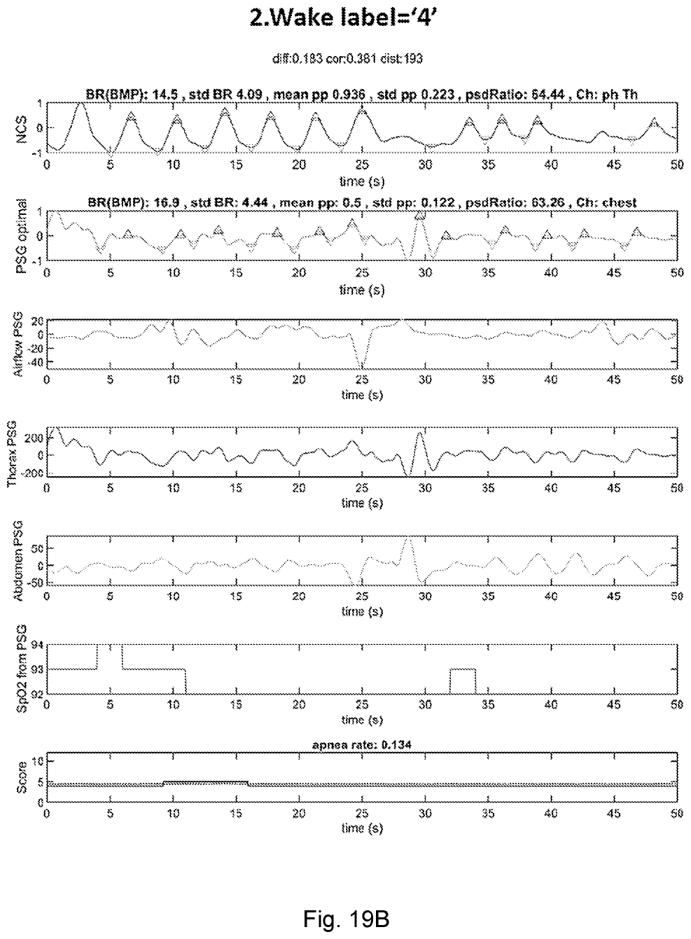

An experiment was conducted to classify a sleep stage. Examples of PSG data taken during a sleep stage (N1) and a wake state are shown in FIGS. 19A and 19B, respectively. A machine learning classifier was trained using mean breathing rate, breathing rate standard deviation, breathing rate coefficient of variation (COV), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, Power Ratio, breathing cycle number (total number of detected breathing cycles in the specific epoch), and time duration that no peak is detected, mean blood oxygen level, standard deviation of the blood oxygen level, percentage of time blood oxygen level is greater than a threshold value (e.g., a predetermined threshold value), and mean skewness of the breathing rate.

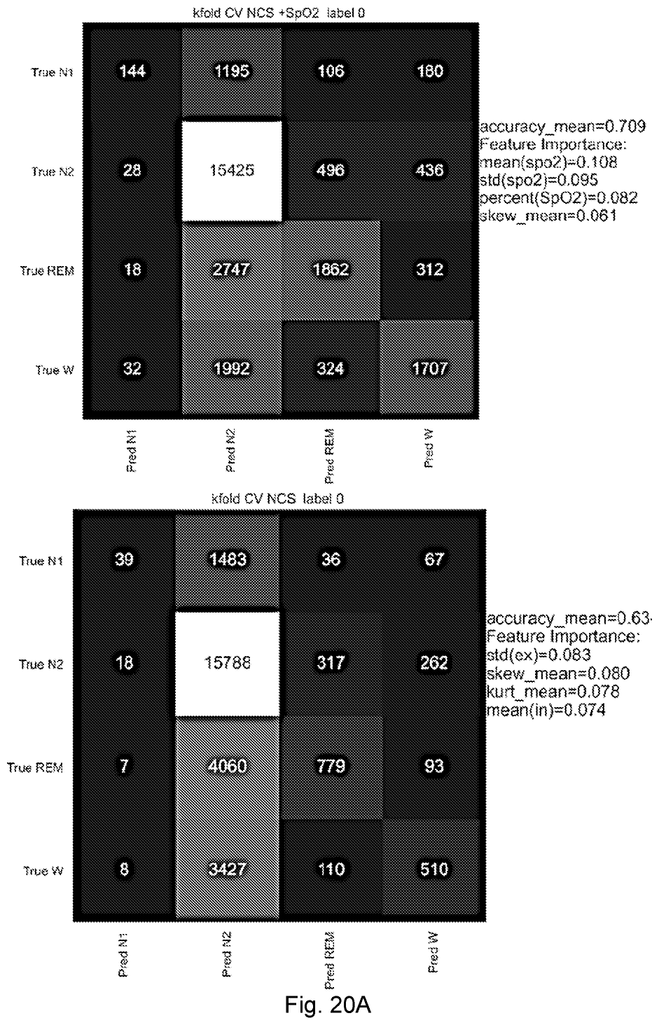

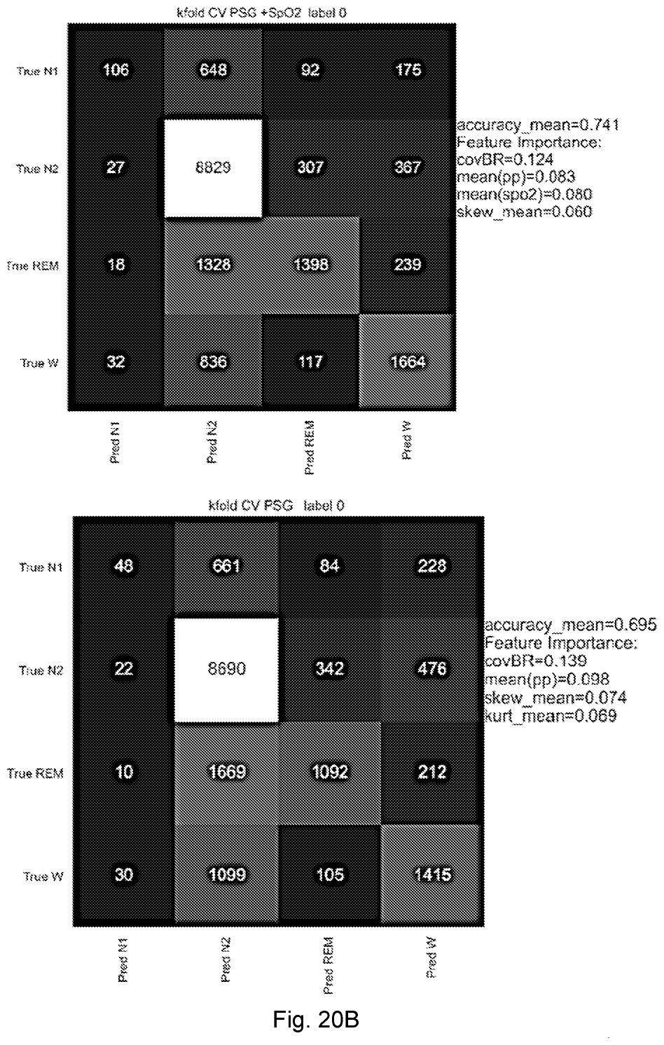

In a first example, the classifier was trained to classify the individual's sleep stage as non-REM 1 (N1), non-REM 2 (N2), REM, or “wake.” Results are shown in FIG. 20A (using NCS data) and FIG. 20B (using PSG data). In each figure, the results shown at the top were generated using SpO₂ features, while the results shown in the bottom portion were generated without SpO₂ features.

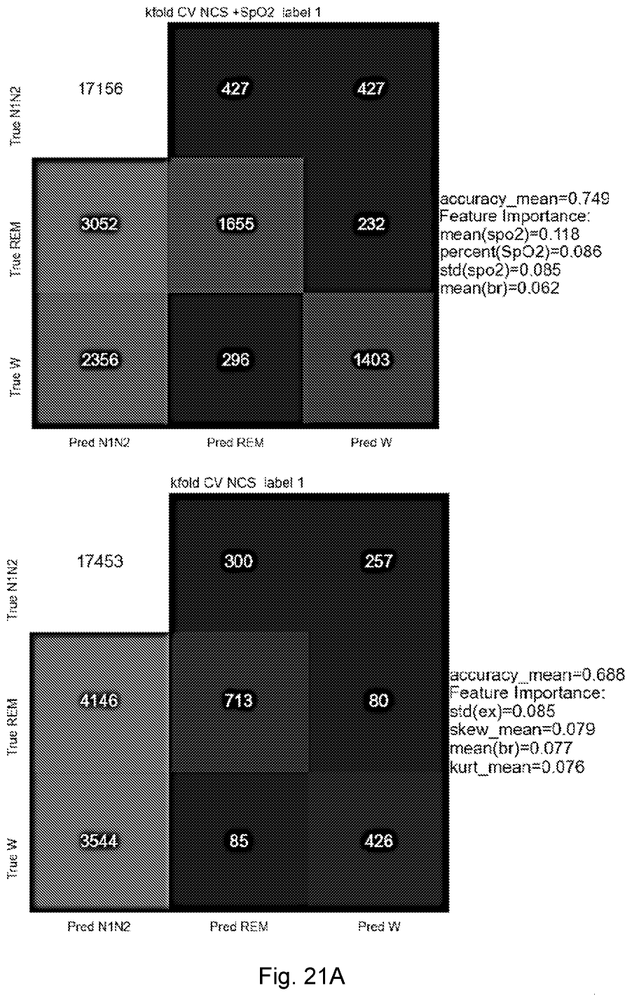

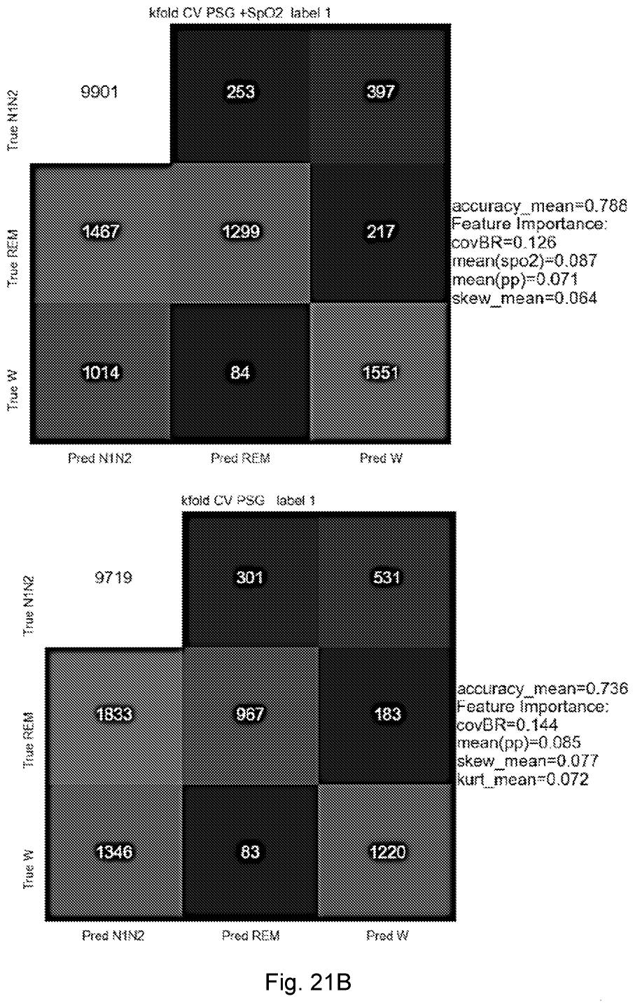

In a second example, sleep stages N1 and N2 were combined. As such, the classifier was trained to classify the individual's sleep stage as N1N2, REM, or “wake.” Results are shown in FIG. 21A (using NCS data) and FIG. 21B (using PSG data). In each figure, the results shown at the top were generated using SpO₂ features, while the results shown in the bottom portion were generated without SpO₂ features.

Although the present disclosure has been described with respect to one or more particular embodiments, it will be understood that other embodiments of the present disclosure may be made without departing from the spirit and scope of the present disclosure.

1. A method for monitoring respiration of an individual, comprising:
providing a first radiofrequency (“RF”) sensing signal within a near-field coupling range of a respiratory motion to be measured to generate a respiratory measurement signal as the first RF sensing signal modulated by the respiratory motion; detecting the respiratory measurement signal;
measuring the respiratory motion over a measurement period based on the respiratory measurement signal; and
detecting a respiratory event using the measured respiratory motion.

2. The method of claim 1, further comprising predicting a respiratory event of the individual using a machine learning classifier, wherein the respiratory event is one or more of a central apnea, an obstructive apnea, a mixed apnea, a hypopnea, or respiratory effort related arousal (RERA).

3. The method of claim 2, wherein the classifier is trained using one or more of the following parameters determined from the measured respiratory motion: mean breathing rate, breathing rate standard deviation, breathing rate coefficient of variation (COY), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, power ratio, breathing cycle number, and time duration that no peak is detected.

4. The method of claim 3, further comprising:
providing a second RF sensing signal within a near-field coupling range of a second motion to be measured to generate a second measurement signal;
detecting the second measurement signal; and
measuring the second motion based on the second measurement signal.

5. The method of claim 4, wherein the respiratory motion is a thoracic motion, the second motion is an abdominal motion, and the classifier is further trained using a phase difference between the thoracic motion and the abdominal motion.

6. The method of claim 2, further comprising measuring blood oxygen level of the individual over the measurement period.

7. The method of claim 6, wherein the classifier is trained using one or more of: mean blood oxygen level, standard deviation of the blood oxygen level, percentage of time blood oxygen level is greater than a threshold value, and mean skewness of the breathing rate.

8. The method of claim 2, further comprising providing an alert signal if a respiratory event is predicted.

9. The method of claim 1, further comprising providing an alert signal if a respiratory event characteristic of respiratory failure is detected.

10. The method of claim 1, further comprising:
providing a second RF sensing signal within a near-field coupling range of a second motion to be measured to generate a second measurement signal;
detecting the second measurement signal; and
measuring the second motion based on the second measurement signal.

11. The method of claim 10, wherein the respiratory motion is a thoracic motion, the second motion is an abdominal motion.

12. A system for monitoring respiration of an individual comprising:
a first signal source for generating a first RF sensing signal;
a first antenna in electrical communication with the first signal source and wherein the first antenna is configured to be disposed within a near-field coupling range of a respiratory motion to be measured to generate a respiratory measurement signal as the first RF sensing signal modulated by the respiratory motion;
a first receiver for detecting the respiratory measurement signal; and
a processor configured to detect a respiratory event using the respiratory measurement signal.

13. The system of claim 12, wherein the processor further comprises a machine learning classifier configured to predict a respiratory event of the individual, wherein the respiratory event is a central apnea, an obstructive apnea, a mixed apnea, a hypopnea, or respiratory effort related arousal (RERA).

14. The system of claim 13, wherein the classifier is trained using one or more of the following parameters determined from the measured respiratory motion: mean breathing rate, breathing rate standard deviation, breathing rate coefficient of variation (COV), mean peak-to-peak amplitude, standard deviation of peak-to-peak amplitude, COV of peak-to-peak amplitude, mean inhalation time, inhalation time standard deviation, mean exhalation time, exhalation time standard deviation, skewness of breathing rate, kurtosis of the breathing rate, entropy of the breathing rate, power ratio, breathing cycle number, and time duration that no peak is detected.

15. The system of claim 14, further comprising:
a second signal source for generating a second RE sensing signal;
a second antenna in electrical communication with the second signal source and wherein the second antenna is configured to be disposed within a near-field coupling range of a second motion to be measured to generate a second measurement signal as the second sensing signal modulated by the second motion; and
a second receiver for detecting the second measurement signal.

16. The system of claim 15, wherein the respiratory motion is a thoracic motion, the second motion is an abdominal motion, and the classifier is further trained using a phase difference between the thoracic motion and the abdominal motion.

17. The system of claim 13, further comprising a blood oxygen sensor in communication with the processor.

18. The system of claim 17, wherein the classifier is trained using one or more of: mean blood oxygen level, standard deviation of the blood oxygen level, percentage of time blood oxygen level is greater than a threshold value, and mean skewness of the breathing rate.

19. The system of claim 13, wherein the processor is further configured to provide an alert, signal if a respiratory event is predicted.

20. The system of claim 12, wherein the processor is further configured to provide an alert signal if a respiratory event characteristic of respiratory failure is detected.

21. The system of claim 12, further comprising:
a second signal source for generating a second RF sensing signal;
a second antenna in electrical communication with the second signal source and wherein the second antenna is configured to be disposed within a near-field coupling range of a second motion to be measured to generate a second measurement signal as the second sensing signal modulated by the second motion; and
a second receiver for detecting the second measurement signal.

22.-28. (canceled)
